(19) United States
(12) Patent Application Publication
(10) Pub. No.: US 2021/0106742 A1
(43) Pub. Date: Apr. 15, 2021

(54) NON-HEMOLYTIC COMPOSITIONS AND METHODS OF USE FOR RECOVERING DISEASE CAUSING TOXIC CONSTITUENTS IN THE BLOOD

(71) Applicant: ACTORIUS INNOVATIONS AND RESEARCH CO, Simi Valley, CA (US)

(72) Inventor: Jayant Jagannath KHANDARE, Pune (IN)

(21) Appl. No.: 17/069,277

(22) Filed: Oct. 13, 2020

Related U.S. Application Data
(60) Provisional application No. 62/914,623, filed on Oct. 14, 2019.

(51) Int. Cl. A61M 1/34

(52) U.S. Cl. CPC A61M 1/3468; A61M 1/3431; A61M 1/3472; A61M 2205/75; A61M 2202/005; A61M 2202/0057; A61M 2202/0014

(57) ABSTRACT

The present disclosure relates to non-hemolytic adsorbent compositions useful for isolating, enumerating, accounting, and removing the disease-causing toxic constituents in the blood. The said compositions are useful in identifying the disease, disease status, and validating the efficacy of the therapeutic treatment being administered for the treatment of the disease. Methods for isolating, enumerating, accounting, and removing disease-causing toxic constituents in the blood as well as monitoring the disease status and validating the efficacy of the therapeutic treatment being administered for the treatment of the disease are disclosed.

### CROSS-REFERENCE TO RELATED APPLICATIONS

This application claims priority under 35 USC § 119(e) to U.S. Provisional Patent Application Ser. No. 62/914,623 filed Oct. 14, 2019 and is incorporated in full by reference herein.

### BACKGROUND

Field of the Discovery. The present disclosure relates to non-hemolytic adsorbent compositions and methods useful in the isolation and enumeration of toxic constituents of the blood, especially circulating tumor cells that help in the management of cancer. The invention relates to diagnosis and prognosis for diseases as well as in the screening of therapeutic treatments of diseases, especially oncological diseases. Methods that are suitable for the removal of toxic constituents from the blood of a patient using a closed system are disclosed.

Background Information Cancer has been conventionally regarded clinically as a locoregional disease. However over the past four decades increasing clinical evidence has ascertained that it is indeed a local manifestation of a systemic process. (Zajicek G. Med Hypotheses. 1978; 4(3) 193-207). Cancer therapies have predominantly targeted primary tumorigenesis with increasing success over the past decades. However, efforts to curtail or prevent the spread of cancer to the distant organs have proved challenging. Metastatic disease remains the foremost cause of mortality in the majority of cancer patients. Neoplastic cells such as circulating tumor cells (CTCs) are continuously shed from a primary tumor site and disseminate systemically via the peripheral blood circulation and eventually reach the distant organs, and initiate secondary tumorigenesis or metastasis.

In addition, the cancer patient's blood contains several other toxic cell particles and cell debris, and attempts have been made to detect cancer by isolating and enumerating them. These include but are not limited to CTCs, CTC clusters, cell-free nucleic acids (CfDNA), cancer cell-associated nucleic acids (CtDNA), and exosomes. Amongst these, CTCs are the most active, aggressive, and clinically validated biomarkers that are known to cause metastasis and are directly correlated with over-all survival.

The circulating tumor cells are known to carry vital prognostic information vis-à-vis tumor onset, progression, and metastasis (Habli, Z. et al. Cancers (Basel). 2020; 12(7): E1930). The unique biological characteristics that differentiate CTCs from healthy cells has made them attractive targets for diagnostic and therapeutic purposes in numerous carcinomas. While CTC number in the blood is very low, their presence before and after the treatment directly correlates with progression of metastasis and overall survival (OS) (Cristofanilli, M. et al. N Engl J Med. 2004; 351:781-791; De Bono J. S. et al. Clin. Cancer Res. 2008; 14, 6302-6309). The extravasation and dissemination of CTCs into the peripheral blood originates at the early onset of the primary tumor cell proliferation and tumor formation. The progression of shedding of CTCs simultaneously from such tumor sites paves the seeding for the metastatic spread and is undetected at the early stage of the cancers. Furthermore, such small tumors at an early stage are below the detection limits of imaging techniques such as positron emission tomography (PET).

The variation in the number of CTCs post-chemotherapy and other cancer treatments can alter the disease dynamics with respect to the progression-free survival (PFS) and OS of the patient (Botteri et al. Breast Cancer Res. Treat. 2010; 122, 211-217; Cohen et al. J. Clin. Oncol. 2008; 26, 3213-3221; Hayes et al. Clin. Cancer Res. 2006; 12, 4218-4224; Miller et al. J. Oncol. 2010; 2010:617421).

The short life-span of CTCs due to a multitude of inherent host processes (e.g. shearing forces, anoikis, apoptosis, host immune responses), results in low abundance in the range of 1-10 CTCs compared to billions of blood cells per milliliter of blood of metastatic cancer patients, thereby making accounting of CTC numbers through isolation and separation techniques extremely challenging (Allard et al. Clin. Cancer Res. 2004; 10, 6897-6904). In spite of the low CTC number in the blood that would activate the metastasis progression, the treatments such as radiation, surgery, and chemotherapy, which are primarily focused at the solid tumor tissues do not eliminate the CTCs which are in circulation and are distributed in the blood.

Chemotherapy treatments initiated at an early stage of cancers often exhibit a positive response in tumor regression, however, the tumors are often known to relapse and further result in local or distant metastasis. The overall survival in these cases is substantially reduced. It is recognized that metastasis detection, which involves CTC detection, enumeration and accounting of the phenotype and genotype is important for the treatment decisions. The traits in identifying the origin of cancers with specific markers from blood remains challenging, biomarkers such as epithelial growth factor (EGFR) in the lung, BReast CAncer gene (BRCA) 1 and BRCA 2 in the breast cancer have been validated and more markers need validation.

Numerous CTC enrichment methods based on biological, chemical, and physical properties of CTCs are reported for their isolation, enumeration, and finally detection. These can be classified into size-based separation (Farace et al. Br. J. Cancer 2011, 105, 847-853), density-based separation (Weitz et al. Clin. Cancer Res. 1998; 4, 343-348), surface-charge based separation (Gascoyne et al. Electrophoresis 2009; 30, 1388-1398), and microfluidic device separations (Hur et al. Lab on a Chip 2011; 11, 912-920).

Affinity-based immunomagnetic separation is one of the most commonly used methods based on unique biological expression patterns exhibited exclusively by CTCs but not normally manifested in healthy individual's cells. In this technique, CTCs are captured by using ligands (e.g., anti-Epithelial Cell Adhesion Molecule (EpCAM) antibody) which bind to specific biomarkers which are over-expressed on the CTCs of epithelial cancers of origin. In immunomagnetic separation, magnetically active substrates are conjugated with one or more complementary antibodies that specifically bind to immunogenic biomarkers of CTCs (Balic et al. Expert Rev. Mol. Diagn. 2012, 12, 303-312). CellSearch markets a test to enrich and enumerate CTCs from the metastatic breast, prostate, and colorectal cancer patient's blood is based on immunomagnetic separation that uses the anti-epithelial cell adhesion molecule antibody (Miller et al. J. Oncol. 2010, 617421. (U.S. Pat. Nos. 7,901,950, 8,337,755, US 2013/0157347). Similarly, anti-epithelial cell adhesion molecule antibody based immunomagnetic material OncoViu® CTC Diagnostic Test kit has been recently approved by CDSCO/Drug Controller General of India for capturing CTCs from epithelial origin cancers including breast, lung, head, and neck cancers (Medical Devices Rules, DCGI/CDSCO India 2019).

A crossflow module for the removal of CTCs by using cell size exclusion filters; was reported (US 2015/0041398). Methods to isolate CTCs from blood-based on size-based filtration, immuno-selective interaction have also been reported. US 2013/039707 describes the capture of CTCs using filters containing micro-posts. US 2013/0131423 describes the separation of tumor cells by porous filters and adsorbents coated with antibodies. US 2014/0074007 describe the separation of tumor cells by porous membrane filters and a support material coated with antibodies. US 2011/0244443 and US 2015/0041398 describes the separation of tumor cells by size-based filtration using porous membrane filters as well as by membranes coated with antibodies. Viatar CTC solution explored CTCs capture for increasing the overall survival of cancer patients. US 20160058937 and US 20150121808 describe the capture of CTCs by micropillars and microbeads coated with antibodies. These methods report the separation of other cancer-causing components such as circulating cell-free tumor DNA (ct-DNA), exosomes, Epithelial-Mesenchymal Transition (EMT) cells (a subset of metastatic progenitor cells/CTCs, expressing alternative/mesenchymal cellular markers), and micro-RNA (mi-RNA). Khandare et al., showed the immunomagnetic enrichment of CTCs by glass capillaries coated with Fe₃O₄ nanoparticles conjugated to transferrin ligand (Adv. Mater. Interfaces, 2017, 4, 1-9) and with Fe₃O₄ nanoparticles conjugated to the anti-epithelial cell adhesion molecule antibody (EP 3259598).

Early CTC detection with increased efficiency and specificity still remains challenging. In general, the current methods using adsorbents for cell capture are time-consuming, expensive, non-specific, and less efficient, in particular, when large volumes of blood samples for cell filtration are involved. For example, in the case of size-exclusion membrane filters—the small-sized pores in removing leukocytes and platelets can be clogged by large-sized cells such as CTCs and normal epithelial cells.

Furthermore, as a result of the time elapsed in removing CTC like cells, there is a higher possibility of destruction of cells which makes their determination inaccurate. As a result, the treatment administered would be less effective.

U.S. Pat. No. 6,190,870 shows immunomagnetic isolation followed by flow cytometric enumeration. However, before immunomagnetic separation, the blood samples are pre-processed using density gradients. Furthermore, there is no discussion on isolating or counting anything other than intact cells. There is no visual analysis of the samples.

U.S. Pat. No. 6,197,523 described the enumeration of cancer cells in 100 microliter blood samples. The methods used capillary microscopy to confirm the identity of cells that were found. The methods are specific for intact cells, and there is no discussion of isolating or enumerating fragments or debris.

U.S. Pat. No. 6,365,362 described methods for immunomagnetically enriching and analyzing samples for tumor cells in the blood. WO 02/20825 described use of an adhesion matrix for enumerating tumor cells. The matrix is analyzed for the presence and accounting of the type of captured cells.

CTC's degradation and debris formation are known to confound the detection of CTCs by direct enrichment procedures from whole blood. The number of intact CTCs, damaged or suspect CTCs as well as the degree of damage to the CTCs, further serve as diagnostically important indicators of the tumor burden, the proliferative potential of the tumor cells, and/or the effectiveness of therapy. The methods and protocols of the prior art lead to unavoidable in vivo damage to CTCs due to longer processing time and damage of cells, thus resulting in the erroneous outcome for the realistic tumor burdens thereby resulting in sub-optimal treatment response in cancer patients. As such, there remains an ongoing need in the art for novel compositions and methods for the detection of CTCs.

### SUMMARY

There is no method presently available for the simultaneous CTC detection, enumeration, and minimizing the CTC and CTC clusters by concentrating them and then removing them from the blood, especially because there are no non-hemolytic adsorbent compositions reported in the art which can be used for separating, quantifying, and recovering disease-causing toxic constituents in the blood.

The development of such non-hemolytic adsorbent compositions will be useful in developing a relatively simple blood test, which shows high sensitivity and specificity, and offer a “real-time liquid biopsy’, followed by the removal CTCs as a cancer treatment for early as well late stage cancers.

Various embodiments of the present invention disclose compositions, and methods that at least partly address the limitations of the prior art.

According to an embodiment of the present invention are provided non-hemolytic compositions comprising paramagnetic substrates as exemplified by iron oxide.

According to an embodiment of the present invention are provided non-hemolytic compositions comprising non-paramagnetic substrates as exemplified by glass.

According to an embodiment of the present invention are provided non-hemolytic compositions comprising paramagnetic substrates as exemplified by iron oxide which is cross-linked.

According to an embodiment of the present invention are provided non-hemolytic compositions comprising non-paramagnetic substrates as exemplified by glass which is cross-linked.

According to an embodiment of the present invention are provided non-hemolytic compositions comprising functionalizing agents selected from glutathione, cysteine, citric acid, (3-aminopropyl)triethoxysilane (APTES), (3-glycidyloxypropyl)trimethoxysilane (GPTMS), (3-glycidyloxypropyl)triethoxysilane (GPTES), (3-mercaptopropyl)trimethoxysilane (MPTMS), (3-mercaptopropyl)triethoxysilane (MPTES).

According to an embodiment of the present invention are provided non-hemolytic compositions comprising spacers selected from glutathione, citric acid, silanes, polymers-dendrimers, hyper branched polymers.

According to an embodiment of the present invention are provided non-hemolytic compositions comprising ligands selected from anti-epithelial cell adhesion molecule antibody (anti EpCAM), proteins (transferrin, Bovine Serum Albumin (BSA)), carbohydrate ligands (N-acetyl glucosamine (NAG).

According to an embodiment of the present invention are provided non-hemolytic compositions which can be sterilized, in whole, by using irradiation, solvents and ethylene oxide, prior to use.

According to an embodiment of the present invention are provided non-hemolytic compositions which can be sterilized, in part, by using irradiation, solvents, and ethylene oxide, prior to use.

According to an embodiment of the present invention are provided non-hemolytic compositions comprising a ligand which bind to constituents selected from, circulating tumor cells, proteins, drugs, nucleic acids, cell debris, peptides, cell free DNA, exosomes, Epithelial-mesenchymal transition (EMT) cells, micro-RNA (mi-RNA) and other materials that is desired to be removed from blood of a cancer patient.

According to an embodiment of the present invention are provided non-hemolytic compositions comprising a ligand which are useful recovering from blood, drugs selected from Vancomycin, Metformin, Doxorubicin, Methotrexate, Paclitaxel, 5 Fluorouracil, Cisplatin, and Camptothecin, Docetaxel, Oxaliplatin, Cyclophosphamide.

According to an embodiment of the present invention are provided non-hemolytic compositions comprising a ligand which is selected from anti-bodies, peptides, proteins, chemotherapy agents, ionic molecules, carbohydrates, and biomarkers.

According to an embodiment of the present invention are provided non-hemolytic compositions which capture CTCs from cancer patients' bloods using ligands exemplified by anti-epithelial cell adhesion molecule antibody at efficiency greater than 85%.

According to an embodiment of the present invention are provided non-hemolytic compositions which are useful in isolating, enumerating, accounting, imaging, removing constituents selected from, circulating tumor cells, proteins, drugs, nucleic acids, debris, peptides, and other material that is desired to be removed from blood of a cancer patient.

According to an embodiment of the present invention are provided non-hemolytic compositions which are useful in removing circulating cancer cells from the blood of a cancer patient and thereby minimizing/inhibiting/eliminating cancer metastasis.

According to an embodiment of the present invention, are disclosed methods of synthesis of non-hemolytic compositions.

According to an embodiment of the present invention, are disclosed methods which provide a real time, non-invasive, and extracorporeal liquid biopsy.

According to an embodiment of the present invention, are disclosed methods to diagnose, monitor, screen and treat disease, evaluate the effectiveness of a therapy based on measurement of circulating rare cells, including malignancy as determined by CTC, clusters, fragments, and cell debris.

According to an embodiment of the present invention are disclosed methods to isolate tumor cells and use them culturing to study the genotypic changes of the tumor, tumor markers, overexpression of genetic mutation, aiding to study the drug treatment reposes before the start of the treatment and anticipate of the advance drug/s resistance.

According to an embodiment of the present invention the separation of circulating tumor cells using non-hemolytic compositions using affinity-based separation avoids clogging effect as well as non-specific separation of various vital blood components that is associated with pore/membrane-based CTC enrichment.

According to an embodiment of the present invention are disclosed methods useful in assessing a favourable or unfavourable over-all survival, progression free survival and even averting the chemotherapy that could result in serious drug side-effects especially when the prognosis is favourable. Thus, the present invention can be used for prognosis of any of a wide variety of disorders relating to epithelial and endothelial cell enumeration.

According to an embodiment of the present invention, the cultured cells from cells isolated using non-hemolytic compositions are the true phenotype and genotype with bio-signatures and allows rare cells to scale up for better clinical validations which can be more predictable and accurate. These cultured cells can be used for assessing drug response, drug resistance, therapy choices and can be used for developing personalized immunological cancer treatments including Chimeric Antigen Receptor T cells (CAR-T) for the said patient.

Those skilled in the art will be able to design numerous non-hemolytic compositions using the methods described herein which would achieve capture and elimination of other cancer causative or non-hematopoietic substances from the blood (e.g. CfDNA, ctDNA, exosomes, drugs, miRNA).

### BRIEF DESCRIPTION OF THE DRAWINGS

FIG. 1 Iron oxide nanoparticles of example 1 size analysis using Transmission Electron Microscopy.

FIG. 2 Iron oxide nanoparticles of example 1 exhibiting paramagnetic property.

FIG. 3 Microscopy image of crosslinked glutathione functionalized iron oxide particles of example 14.

FIG. 4 Crosslinked glutathione functionalized iron oxide particles of example 14 exhibiting paramagnetic property.

FIG. 5 Crosslinked (3-aminopropyl)silane functionalized glass beads using glutaraldehyde of example 16.

FIG. 6 (i) Physical absorption of glutathione functionalized iron oxide nanoparticles on (3-aminopropyl)silane functionalized glass beads; (ii) Crosslinking of glutathione functionalized iron oxide nanoparticles with (3-aminopropyl)silane functionalized glass beads of example 17.

FIG. 7 Shows the change in colour after crosslinking glutathione functionalized iron oxide nanoparticles with glass beads (compare (i) and (ii)), which are crosslinked (3-aminopropyl)silane functionalized glass beads with glutathione functionalized iron oxide nanoparticles of example 21.

FIG. 8 (i) Microscopy image of crosslinked glutathione functionalized iron oxide nanoparticles with (3-aminopropyl)silane functionalized glass coverslip of example 21, (ii) Microscopy image of (3-aminopropyl)silane functionalized glass beads of example 10. (iii) Magnified microscopy image of the crosslinked glutathione functionalized iron oxide nanoparticles with (3-aminopropyl)silane functionalized glass coverslip of example 21.

FIG. 9 (i) Fluorescence image of fluorescein isothiocyanate tagged to glass bead of example 28; (ii) Fluorescence image of fluorescein isothiocyanate tagged to glass beads of example 11.

FIG. 10 (i) Fluorescence image of fluorescein isothiocyanate tagged to glass beads of example 30; (ii) Fluorescence image of fluorescein isothiocyanate tagged to glass beads of example 12.

FIG. 11 (i) Fluorescence image of fluorescein isothiocyanate tagged to glass beads of example 31; (ii) Fluorescence image of fluorescein isothiocyanate tagged to glass beads of example 12.

FIG. 12 Circulating Tumor Cells capture in a series of trap device using cancer patient whole blood using composition of example 24.

FIG. 13 Circulating Tumor Cells capture in a series of trap device using cancer patient whole blood divided in equal volumes 1 milliliter using composition of example 24.

FIG. 14 Human colon cancer (HCT-116) cell line capture with composition of example 28 (glass cover slip).

FIG. 15 Human colon cancer (HCT-116) cell line capture with composition of example 28 (glass beads).

FIG. 16 Circulating tumor cell capture from cancer patient blood with composition of example 24 in a glass capillary tube.

FIG. 17 Circulating tumor cell capture from cancer patient blood with composition of example 25.

FIG. 18 Capture of circulating tumor cell cluster from cancer patient blood with composition of example 28 (glass cover slip).

FIG. 19 Circulating tumor cell capture from cancer patient blood with composition of example 30.

FIG. 20 Circulating tumor cell capture from cancer patient blood with composition of example 33.

FIG. 21 Circulating tumor cell capture from cancer patient blood with composition of example 35.

FIG. 22 Circulating tumor cell capture from cancer patient blood with composition of example 45.

FIG. 23 Circulating tumor cell capture and cell debris from cancer patient blood with composition of example 49.

FIG. 24 Circulating tumor cell capture from cancer patient blood with composition of example 51.

FIG. 25 Circulating tumor cell capture from cancer patient blood with composition of example 52.

FIG. 26 Circulating tumor cell capture from cancer patient blood with composition of example 53.

### DETAILED DESCRIPTION

It is well established that the cancers of epithelial origin such as breast, colon, rectal, lung, head and neck, are organ-confined diseases in its early stages. The primary objective and focus of treatment and chemotherapy are limited by incomplete primary tumor regression leading to metastasis. Currently, tumor is confirmed by the Computed Tomography/Positron Emission Tomography (CT/PET) imaging tools followed by the solid tissue biopsy and histopathology, which are often limited due to tumor inaccessibility and invasiveness. The sensitivity of CT/PET imaging is limited up to 5 mm sized tumors, and the lesions smaller than 5 mm can be missed although they can accommodate trillions of cancer cells. Confirmation of tumor absence, tumor regression, or disease free or complete cancer cure is considered as a positive tumor response arrived at based on the above techniques is often misleading due to the lower sensitivity of the above tools.

Such patients are referred for the follow-ups receive next treatment only after until the CT/PET images show the presence of the tumor, metastasis, and the relapse of the disease. This is often detrimental for the next line of chemotherapy and in the majority of times, it correlates with a prediction of short survival of the patient.

There is now well-established evidence that the primary cancer organs harbour and shed cancer cells and reach the peripheral blood circulation even at an early disease stage prior to the appearance of clinical manifestations. These cells are termed as ‘circulating tumor cells’ (CTCs). Upon tumor vascularization, these cells disseminate and shed into the circulation and upon colonization at distant sites to form metastases. Often, CTCs shred and extravagate in the form of CTC clusters which are known to be aggressive in translating distant tumor metastasis. However, chemotherapy cannot destroy CTCs in blood circulation due to its sub-cellular cytotoxicity drug concentration in whole blood. The presence of circulating tumor cells in blood circulation can be used for the percentage prediction of a short survival, treatment response, prognostic decisions with real-time monitoring of the patient. Furthermore, CTCs can be used to screen for cancer sites in concurrence with other tests, such as CT/PET imaging, biopsy, histopathological staining, mammography, or other blood biomarkers such as cell-free nucleic acids (CfDNA, CtDNA), mutations like Epidermal growth factor receptor (EGFR), BReast CAncer gene (BRCA), and prostate serum antigen (PSA).

CTCs are the most active, aggressive, and clinically validated blood biomarkers that are known to cause the metastasis and represent correlation in predicting the decreased percentage of over-all survival. The circulating tumor cells contain overexpression of tumor biomarkers not normally expressed in healthy individual's cells. CTCs account overall survival in all cancer stages and evidence for its metastasis progression can be used for both for diagnostics as well for cancer treatment by detecting, isolating, enumerating, and finally discarding them.

Considering the increasing evidence supporting the prognostic correlation of CTC number and metastatic disease in numerous carcinomas, it would be desirable to eliminate CTCs from patient's blood as to reduce/prevent overt metastatic progression and the occurrence of minimal residual disease (MRD), which would result in enhancing the patient's overall survival (Pantel, K., et al. Nat Rev Clin Oncol. 2019, 16, 409-424; Scarberry, K. E. et al. Nanomedicine. 2011, 6:1, 69-78; Azarin S., et al. Nat Commun. 2015, 6, 8094). (Cohen et al. J. Clin. Oncol. 2008, 26, 3213-3221). Consequently, the isolation, enumeration, accounting and removal of CTCs, as well as other cancer-causing entities, from the blood such as cell-free nucleic acids (CfDNA), cancer cells associated with nucleic acids (CtDNA), exosomes, and chemical entities such as undesirable quantities of drugs would lead to increase progression-free survival and overall survival of cancer patients.

Also in incidences where such cancer cells can be isolated and detected when there are no clinical symptoms or signs of a tumor, it may be possible to identify CTCs presence and correlate the cancer phenotype, genotype, and the organ of origin. Furthermore, CTC numbers in the blood can be used to estimate the disease state, stage, and tumor burden. The higher the CTC cell count per milliliter in blood, greater is the tumor disease burden and unfavorable response to the anticancer treatment. It also indicates the aggressive metastasis signature. Thus monitoring of CTCs, after the surgery or radiation therapy, would be useful in assessing the response to the treatment for follow-up and also in chemotherapy treatment.

Detection of tumor cells in the peripheral blood of patients has the potential not only to detect a tumor at an earlier stage but also to provide clinical indications as to the potential invasiveness of the tumor. The patients with very low or absence of CTCs in the blood may represent localized tumors with less aggression as well the CTC dissemination. Accordingly, the patient may be advised for the next CTC tests as it offers a non-invasiveness method and less exposure to CT/PET radiations.

Clusters of CTCs are more likely to lead to secondary cancers as compared to that caused by the single CTCs. Hence diagnosing the presence of clusters is highly critical, as their presence indicates the rapid onset of metastasis.

Magnetic materials comprising affinity ligands have been reported in the prior art for the isolation, enumeration and accounting of circulating tumor cells (CTCs), CTC clusters, cell-free nucleic acids (CfDNA), cancer cells associated nucleic acids (CtDNA) and exosomes. The recent patent EP 3259598 which forms the basis of a regulatory approved OncoViu technology consisted of (1) transferrin (2) iron oxide nanoparticles, (3) cyanine N-hydroxysuccinimide 5 dye, (4) fourth-generation (G4) dendrimers (5) glutathione (GSH), These compositions are to be used for isolation and detection of cancer cells from blood using red blood lysis buffers, as an in vitro diagnostics method. They cause hemolysis and thus cannot be used for the removal of cancer cells after which the blood can be returned to the patient body.

The compositions for the removal of CTCs, CTC clusters, cell-free nucleic acids (CfDNA), cancer cells associated nucleic acids (CtDNA) and exosomes must be non-hemolytic. Further it would be desirable that the same composition be used for the isolation, enumeration accounting and removal of circulating tumor cells (CTCs), CTC clusters, cell-free nucleic acids (CfDNA), cancer cells associated nucleic acids (CtDNA) and exosomes, after which the blood is returned to the patient body.

It has now been surprisingly found that compositions comprising affinity ligands but lesser number of constituents than those disclosed in EP 3259598 can be obtained which are non-hemolytic and which can be used for the isolation, enumeration accounting and removal of circulating tumor cells (CTCs), CTC clusters cell-free nucleic acids (CfDNA), cancer cell-associated nucleic acids (CtDNA) and exosomes, after which the blood can be returned to the patient body.

As has been mentioned above magnetic materials have been chosen in the past as substrates to which affinity ligands are linked.

Presently described are compositions comprising iron oxide as a paramagnetic substrate and/or glass beads as non-paramagnetic substrate to which ligands are linked using the methods disclosed herein.

An advantage of glass substrate is that it is non-hemolytic. Further, compositions comprising glass beads can be separated by gravity separation.

In any of aspect or embodiment described herein, the non-hemolytic compositions comprise a ligand and a functionalized substrate. In any of aspect or embodiment described herein, the substrate is paramagnetic as exemplified by iron oxide. In any of aspect or embodiment described herein, the substrate is not paramagnetic, for example, glass.

For instance it is reported that the iron oxide nano particles smaller than 10 nm can bind nonspecifically to the constituents of the blood. U.S. Pat. No. 7,863,012 states that the iron oxide nano particles less than about 200 nm behave as colloids. Iron oxide nano particles of different particle sizes can be mixed to achieve optimal performance.

In any of aspect or embodiment described herein, magnetic iron oxide particles of the particle size around 10 nanometers are further crosslinked to obtain iron oxide particles, which results in effective size of the crosslinked particles. The crosslinked iron oxide particles have the advantage of larger surface area and, at the same time, minimize nonspecific binding to the constituents of the blood, and do not interfere during imaging.

In any of aspect or embodiment described herein, the non-hemolytic compositions comprise optionally a spacer. In any of aspect or embodiment described herein, the non-hemolytic compositions comprise a cross linking agent.

A wide range of non-hemolytic compositions useful as adsorbents can be synthesized 1) by reacting functionalized substrates with spacers, 2) by reacting with spacers followed by reaction with ligands, 3) by crosslinking functionalized substrates followed by the reaction with spacers followed by reaction with biocompatible ligands, 4) by functionalizing the substrates followed by crosslinking with either the same functionalized substrates or another functionalized substrate followed by reacting with ligands.

As used herein, the term ‘functionalizing agent’ refers to a molecule that attaches itself to a substrate through a functional group but there is no covalent linkage between the two.

As used herein, the term ‘spacer’ refers to a molecule that is covalently linked to the functionalizing agent at one end and covalently linked to the ligand at another end.

As used herein, the term ‘ligand’ refers to a molecule that is covalently linked to the spacer at one end and non-covalently attaches itself to the moiety in the blood which is to be at least one of isolated, enumerated, accounted, or removed.

In any aspect or embodiment described herein, a molecule, which acts a ‘functionalizing agent’ in one embodiment may act as a ‘spacer’ in another embodiment, when it covalently reacts at both ends. In certain aspects or embodiments described herein, a molecule, which acts a ‘functionalizing agent’ in one embodiment may act as a ligand in another embodiment when it is non-covalently attached to the substrate at one end as well as non-covalently attached to the moiety in the blood which is to be at least one of isolated, enumerated, accounted, or removed.

In any aspect or embodiment described herein, a ‘functionalizing agent’ in one embodiment may act as a ‘crosslinking agent’ in another embodiment, wherein it is attached to one substrate non-covalently but another substrate covalently.

In any aspect or embodiment described herein, a molecule which acts as a spacer in one embodiment may act as a ligand in another embodiment, wherein it is linked to the functionalizing agent covalently but attaches itself non-covalently to the moiety in the blood which is to be at least one of isolated, enumerated, accounted, or removed.

In any aspect or embodiment described herein, the functionalizing agent is selected from glutathione, cysteine, citric acid, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), 12-aminododecanoic acid, poly(amidoamine) (PAMAM) or a combination there.

In any aspect or embodiment described herein, the spacer is selected from glutathione, cysteine, citric acid, succinic acid, iminothiolane, 12-aminododecanoic acid, poly(amidoamine) (PAMAM) dendrimer, glutathione, iminothiolane or a combination thereof.

In any aspect or embodiment described herein, the ligand is selected from anti-epithelial cell adhesion molecule antibody, transferrin, bovine serum albumin, N-acetyl glucosamine, glutathione, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), 12-aminododecanoic acid, poly(amidoamine) (PAMAM) or a combination thereof.

In any aspect or embodiment described herein, the crosslinking agent is selected from glutaraldehyde, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), glutathione and iminothiolane.

The meanings of these terms are therefore to be understood in the context of the composition of the non-hemolytic compositions described in examples 1-53 and others which can be synthesized based on the reaction sequences described and taught herein.

Hemolysis Assay

Red Blood Cells from whole human blood were separated by centrifugation at room temperature and washed in sodium chloride solution two times and resuspended in phosphate buffer saline with pH 7.4. The resuspended red blood cells were incubated with the composition to be evaluated for 30 minutes at 37° C. The samples were centrifuged and the supernatant collected and analyzed for hemolysis using UV spectroscopy. The percentage of hemolysis was estimated against a negative control phosphate buffer saline with pH 7.4 and positive control 0.5% Triton-X100 prepared in phosphate-buffered saline with pH 7.4.

Recovery of Drugs

Glutathione functionalized iron oxide nanoparticles linked to bovine serum albumin were incubated with aqueous solutions containing known concentrations of 1) vancomycin hydrochloride 2) metformin hydrochloride up to 12 hours. The nanoparticles were separated by magnetic separation. The supernatants containing unbound drug were analyzed by UV-Vis spectrophotometry and the amounts of respective drugs in the supernatant were estimated. The amount drug bound to the nanoparticles was estimated by difference.

Above experiment was also repeated with (3-aminopropyl)silane functionalized glass beads linked to the spacer glutathione followed by the ligand transferrin. The glass beads were separated by gravity separation.

Recovery of Bovine Serum Albumin

Glutathione crosslinked iron oxide nanoparticles linked to N-acetyl glucosamine were incubated up to 12 hours with aqueous bovine serum albumin solution of known concentration. The particles were separated by a magnet. The supernatant containing unbound bovine serum albumin was analyzed by UV-Vis spectrophotometry and the amount of bovine serum albumin bound to these iron oxide nanoparticles was estimated by difference.

(3-aminopropyl)silane functionalized glass beads cross linked with glutathione and linked to N-acetyl glucosamine were incubated up to 12 hours with aqueous bovine serum albumin solution of known concentration. Glass beads were separated by gravitation. The supernatant containing unbound bovine serum albumin was analyzed by UV-Vis spectrophotometry and the amount of bovine serum albumin in the supernatant was estimated. The amount of bovine serum albumin bound to glass beads was estimated by difference.

Recovery of Deoxyribonucleic Acid

Citric acid functionalized iron oxide nanoparticles linked to poly(amidoamine) dendrimer ligand were incubated for up to 12 hours with aqueous solution of known concentration of deoxyribonucleic acid. These iron oxide nanoparticles were then separated using a magnet. The supernatant containing unbound deoxyribonucleic acid was analyzed by UV-Vis spectrophotometry and the amount of deoxyribonucleic acid in the supernatant was estimated.

Amount of Deoxyribonucleic Acid Bound to Iron Oxide Nanoparticles was Estimated by Difference

Similar experiment was repeated for glass beads functionalized with (3-aminopropyl)triethoxysilane (APTES) and linked to polyamidoamine dendrimer. The glass beads were separated by gravity.

Human Colon Cancer Cells (HCT116) Capture

HCT116 cells were incubated with glass substrate linked with anti-epithelial cell adhesion molecule antibody for 5 minutes. The cells were isolated using a magnet and were enriched and fixed and stained with antibodies against cytokeratin (CK-18) and leucocyte common antigen (CD 45) and counterstained with 4′,6-diamidino-2-phenylindole (DAPI). Isolated cells were imaged using fluorescence microscopy and were characterized as cell that are CK-18⁺, DAPI⁺ and CD45⁻.

HCT116 cells aqueous medium were incubated with (3-glycidyloxypropyl)silane functionalized glass beads linked with anti-epithelial cell adhesion molecule antibody for 5 minutes. The cells were isolated by gravity separation of glass beads.

The Capture of Circulating Tumor Cells (CTC) from Cancer Patient's Blood

Cancer patient's blood was incubated for 12 hours with glutathione functionalized iron oxide nanoparticles linked to transferrin. CTCs bound to iron oxide nanoparticles were isolated using magnet. The captured circulating tumor cells were fixed and stained with antibodies against cytokeratin (CK-18) and leucocyte common antigen (CD45) and counterstained with 4′,6-diamidino-2-phenylindole (DAPI). Isolated cells were imaged using Zeiss fluorescence microscopy and were characterized as cells that are CK-18⁺, DAPI⁺ and CD45⁻.

Cancer patient's blood was incubated with (3-glycidyloxypropyl)silane functionalized glass beads linked to anti-epithelial cell adhesion molecule antibody. CTCs attached to glass beads were isolated by gravity separation.

The compositions and methods described herein offer significant advantages, including, for example, the compositions are non-hemolytic. Also, the cross-linked iron oxide compositions offer inter particle network, optimized size, increased surface area, for incorporating larger number of ligands and thereby higher interactions with cancer cells. In addition, the cross-linked iron oxide compositions minimize non-specific interactions with the constituents of the blood and also do not interfere with the imaging of the cells. The compositions and methods described herein also offer, cells and clusters isolation and utility as a diagnostics, recovery of chemicals and drugs from the whole blood, removal and destruction of cancer cells as a cancer treatment. Significantly, the compositions are efficient even at low concentrations and scalable for the use in cancer diagnostics, recovery, and treatment simultaneously.

The invention is now illustrated with examples which are to be regarded as illustrative in nature and do not limit the scope of invention in any manner.

### Example 1

Preparation of Iron Oxide Nanoparticles by Co-Precipitation Method

1.1 gram iron (II) chloride tetrahydrate (FeCl₂.4H₂O) and 4.0 gram iron (III) chloride hexahydrate (FeCl₃.6H₂O) were dissolved in 75 milliliter distilled water. The pH of the solution was adjusted to 10 by addition of aqueous ammonia solution while continuously stirring the solution. The reaction mixture was then heated at 80° C. in water bath for 30 minutes with continuous agitation. The reaction mixture was cooled to room temperature. The reaction flask was placed on the magnet and iron oxide nanoparticles were allowed to settle and the supernatant was decanted. Fresh distilled water was added to the flask and sonicated to resuspend the synthesized iron oxide nanoparticles. The reaction flask was placed on magnet and the supernatant was decanted. This procedure was repeated until the supernatant became colourless.

Finally, the particles were washed with ethanol and dried in oven at 60° C. and stored as solid.

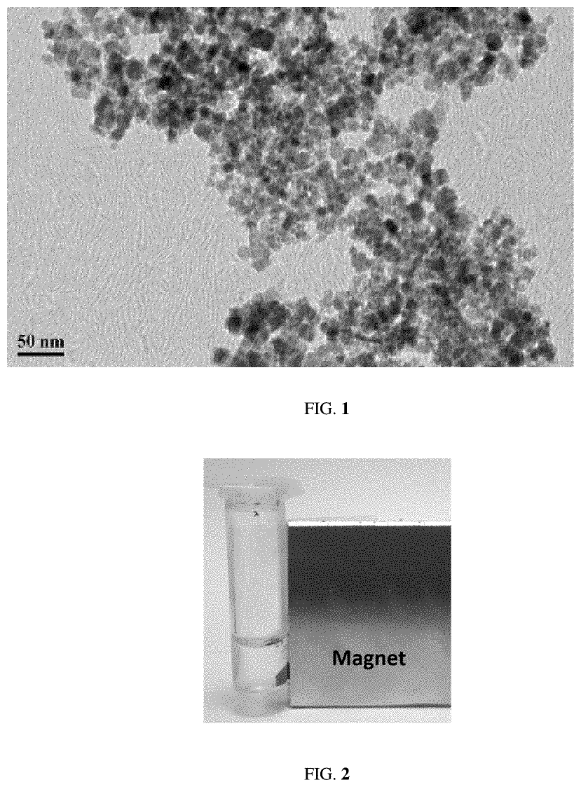

The particle size of synthesized iron oxide nanoparticles was found to be approximately 10 nanometer as determined by Transmission Electron Microscopy (TEM) (see FIG. 1) and the same batch of material was used throughout all following reactions. The synthesized iron oxide nanoparticles were brown in colour and were paramagnetic (see FIG. 2). The spectral region at 3405 cm⁻¹ in Fourier transform infrared spectrum, depicts the presence of hydroxyl groups on the surface of iron oxide nanoparticles while the spectral region from 633 to 579 cm⁻¹ confirmed the presence of Fe—O functionality on iron oxide nanoparticles.

Particles of different shapes and sizes ranging from 10 to 300 nanometer can be synthesized using the same method by varying the temperature, pH, ionic strength or counter anions (Xie W. et al. Shape, size and structure-controlled synthesis and biocompatibility of iron oxide nanoparticles for magnetic theranostics, Theranostics, 2018, 8 (12), 3284).

### Example 2

Functionalization of Iron Oxide Nanoparticles with Glutathione

500 milligram iron oxide nanoparticles of 10 nanometer diameter of example 1 were dispersed in a mixture of 75 milliliter distilled water and 25 milliliter methanol in the reaction flask. 665 milligram glutathione (GSH) was dissolved in 5 milliliter distilled water and added dropwise to the iron oxide nanoparticles dispersion prepared earlier. This reaction mixture was vortexed for 1 hour. The reaction product was then separated magnetically and the supernatant was removed. Additionally, the particles were washed using fresh water until the supernatant was colourless. Finally, the product was washed with ethanol, dried at 60° C. in hot air oven and stored as solid.

The glutathione functionalized iron oxide nanoparticles were brown in colour and were paramagnetic. The functionalization of glutathione onto iron oxide nanoparticles was confirmed by Fourier Transform Infrared Spectroscopy. The spectral region from 1635 to 1400 cm⁻¹ depicts the presence of amide bond of glutathione moiety, confirming the functionalization of glutathione to iron oxide nanoparticles. The spectral peak at 3386 cm⁻¹ represents free carboxylic groups which further validates the presence of glutathione. The ratio of glutathione to iron oxide nanoparticles was varied from 1:1 to 3:1 to obtain iron oxide nanoparticles of varying glutathione content.

### Example 3

Functionalization of Iron Oxide Nanoparticles with Glutathione Using Hydrogen Peroxide.

500 milligram iron oxide nanoparticles of example 1 were dispersed in 90 milliliter distilled water and 665 milligrams of glutathione was added to above mixture. After 30 minutes of stirring, the reaction mixture was transferred to a water bath at 70° C., and 10 milliliter of 30% hydrogen peroxide solution was added dropwise over 1 hour under continuous stirring. The product was separated magnetically and washed with distilled water until the supernatant became colourless. The nanoparticles obtained were dried in an oven at 60° C. and stored as solid.

The glutathione functionalized iron oxide nanoparticles were brown in colour and were paramagnetic. The Fourier transform infrared spectrum with region from 1635 to 1400 cm⁻¹ depicts the presence of amide bond of glutathione moiety, confirming the functionalization of glutathione to iron oxide nanoparticles. The spectral peak at 3386 cm⁻¹ represents free carboxylic groups which further validates the presence of glutathione.

### Example 4

Functionalization of Iron Oxide Nanoparticles with Cysteine

500 milligram iron oxide nanoparticles of example 1 were dispersed in a mixture of 75 milliliter distilled water and 25 milliliter of methanol. 262 milligram cysteine was dissolved in 5 milliliter distilled water and added dropwise to the dispersed iron oxide nanoparticles. The reaction mixture was vortexed for 1 hour. Thereafter the reaction product was separated using magnet and the supernatant was removed. The washing procedure was repeated until the supernatant was colourless. The nanoparticles obtained were dried in oven at 60° C. and stored as solid at 4° C.

The cysteine functionalized iron oxide nanoparticles were brown in colour and were paramagnetic. Functionalization of iron oxide nanoparticles with cysteine was confirmed by Fourier transform infrared spectroscopy which showed peaks at 3432 cm⁻¹, 1618 cm⁻¹, 1418 cm⁻¹ and 637 cm⁻¹ corresponding to carboxylic —OH, —C═O, disulphide —S—S—, and —Fe—O— respectively.

### Example 5

Functionalization of Iron Oxide Nanoparticles with Citric Acid

50 milligram iron oxide nanoparticles of example 1 were dispersed in 20 milliliter distilled water. The pH of the solution was adjusted to 5.0 by dropwise addition of solution of 0.1 gram/milliliter citric acid. The reaction mixture was heated at 95° C. for 1 hour. Thereafter, the reaction product was separated magnetically and supernatant decanted. The washing was repeated successively with 5 milliliter of ethanol and 5 milliliter of distilled water until the supernatant turned colourless. The nanoparticles obtained were dried in oven at 60° C. and stored as solid powder at 4° C.

The citric acid functionalized iron oxide nanoparticles were brown in colour and were paramagnetic. The Fourier transform infrared spectroscopy in the spectral region from 1615 to 1015 cm⁻¹ confirms the presence of carboxylate group (—COO⁻), thereby confirmed the functionalization of iron oxide nanoparticles with citric acid.

### Example 6

Functionalization of Iron Oxide Nanoparticles with (3-Aminopropyl)Triethoxysilane

50 milligram iron oxide nanoparticles of example 1 were dispersed in 100 milliliter toluene in the reaction flask. 75 microliter of (3-aminopropyl)triethoxysilane was added to the iron oxide nanoparticles dispersion. This reaction mixture was vortexed for 12 hours. The reaction product was then separated magnetically and the supernatant was removed. Additionally, the particles were washed with 100 ml distilled water three times. Finally, the product was washed with ethanol, dried at 60° C. in hot air oven and stored as solid at 4° C.

The (3-aminopropyl)silane functionalized iron oxide nanoparticles were brown in colour and were paramagnetic. The Fourier transform infrared spectroscopy in spectral region at 3406 and 1500 cm⁻¹ confirms the presence of amine group (—N—H stretch, —N—H bend), and 1005 cm⁻¹ thereby confirms the presence of —Si—O—Si— (silane functionality) confirmed the functionalization of iron oxide nanoparticles with (3-aminopropyl)silane.

### Example 7

Functionalization of Iron Oxide Nanoparticles with (3-glycidyloxypropyl)trimethoxysilane

50 milligram iron oxide nanoparticles of example 1 were dispersed in 100 milliliter toluene in the reaction flask. 75 microliter of (3-glycidyloxypropyl)trimethoxysilane was added to the iron oxide nanoparticles. This reaction mixture was vortexed for 12 hours. The reaction product was separated magnetically and the supernatant was removed. Additionally, the particles were washed using fresh water with 100 ml distilled water three times. Finally, the product was washed with ethanol, dried at 60° C. in hot air oven and stored as solid at 4° C.

The (3-glycidyloxypropyl)silane functionalized iron oxide nanoparticles were brown in colour and were paramagnetic. The Fourier transform infrared spectroscopy in the spectral region from 1040 to 1093 cm⁻¹ confirms the presence of oxirane group, thereby confirmed the functionalization of iron oxide nanoparticles with (3-glycidyloxypropyl)silane.

### Example 8

Linking of 12-Aminododecanoic Acid Spacer with Citric Acid Functionalized Iron Oxide Nanoparticles

50 milligram of iron oxide nanoparticles of example 5 were dispersed in 20 milliliter 2-(N-morpholino)ethanesulfonic acid buffer pH 6.0. 7.7 milligram N-(3-dimethylaminopropyl)-N′-ethylcarbodiimide hydrochloride and 7 milligram N-hydroxysuccinamide in 2 milliliter 2-(N-morpholino)ethanesulfonic acid buffer pH 6.0 was added to the above iron oxide nanoparticles dispersion and reaction mixture was vortexed for 30 minutes. The reaction product was purified by placing the reaction flask on the magnet and decanting the supernatant. 40 milligram 12-aminododecanoic acid in 1 milliliter phosphate buffered saline pH 7.4 was added to the flask and nanoparticles were sonicated. The dispersed nanoparticles were vortexed for 4 hours. The product was then magnetically separated and the supernatant was removed, followed by washing with 5 milliliter of distilled water. The washing was repeated until the supernatant became colourless. Finally, the product was washed with ethanol, dried at 60° C. in hot air oven and stored in powder form.

The 12-aminododecanoic acid functionalized on citrate iron oxide nanoparticles were brown in colour and were paramagnetic. The Fourier transform infrared spectroscopy which showed spectral peak at 1640 cm⁻¹ confirmed the presence of carboxylate group (—COO⁻) and spectral region at 905-1081 cm⁻¹ confirmed the presence of alkyl groups (—CH₂⁻) thereby confirmed the presence of 12-aminododecanoic acid on citric acid functionalized iron oxide nanoparticles.

### Example 9

Linking of Poly-(Amidoamine) (PAMAM) Dendrimer Spacer with Citric Acid Functionalized Iron Oxide Nanoparticles

50 milligram of iron oxide nanoparticles synthesized as of example 5 were dispersed in 20 milliliter 2-(N-morpholino)ethanesulfonic acid buffer pH 6.0. A solution of 7.7 milligram N-(3-dimethylaminopropyl)-N′-ethylcarbodiimide hydrochloride and 7.0 milligram N-hydroxysuccinamide was added to the above dispersion and stirred for 1 hour. The dispersion was separated using a magnet and washed with phosphate buffer saline pH 7.4. 5 milligram poly(amidoamine) G4 (Aldrich Cat. Number 412449) was added in 1 milliliter of phosphate buffered saline pH 7.4 and reaction mixture was vortexed for 4 hours. The reaction product was separated by placing the reaction flask on magnet and decanting the supernatant. 10 milliliter ethanol was added to the flask and nanoparticles were dispersed. The washing was repeated with 10 milliliter ethanol and 10 milliliter of distilled water alternatively until the supernatant was colourless. The citric acid functionalized iron oxide nanoparticles linked to poly-(amidoamine) were dried in oven at 60° C. and stored at 4° C.

These nanoparticles were brown in colour and were paramagnetic. The Fourier transform infrared spectroscopy which showed spectral peak at 3386 cm⁻¹ confirmed the presence of amine group (—NH₂) and spectral region at 1615-1015 cm⁻¹ confirmed the absence of free citric acid carboxylate groups (—COO⁻) thereby confirmed the linking of citric acid functionalized iron oxide nanoparticles with poly-(amidoamine) (PAMAM). The recovery of deoxyribose nucleic acid was estimated using the procedure described in example 56. The iron oxide nanoparticles without poly(amidoamine) did not capture deoxyribose nucleic acid.

It would be apparent to those skilled in the art that a molecule selected from NH₂-PEG-SH, NH₂—(CH₂)ₙ—COOH (e.g. amino-propionic acid, amino butanoic acid, 10-aminodecanoic acid, 12-aminododecanoic acid), citric acid, (3-aminopropyl)trimethoxysilane (APTES), (3-glycidyloxypropyl)trimethoxysilane (GPTMS), (3-aminopropyl)trimethoxysilane (APTMS), (3-glycidyloxypropyl)triethoxysilane (GPTES), (3-mercaptopropyl)trimethoxysilane (MPTMS), (3-mercaptopropyl)triethoxysilane (MPTES), 3-(trimethoxysilyl)propyl methacrylate, 3-aminopropyl(dimethoxy)ethylsilane, 3-aminopropylmethyldiethoxysilane, amino acid (lysine, alanine), amino acid (natural and artificial), arginylglycylaspartic acid (RGD), polymers (poly(ethylene glycol) (PEG), poly(methacrlyic acid) (PMA), poly (oligo(ethylene glycol) methacrylate, poly(oligo(ethylene glycol)diglycidyl ether, poly(N-isopropyl acrylaminde (PNIPAM), polyimines, poly(amidoamine) (PAMAM), poly(acrylic acid) (PAA), poly(ethylene-co-acrylic acid), poly(lactic acid) (PLA), 2-aminoethoxy acetic acid, peptides molecules with hydroxy groups (eg. Polyvinyl alcohol (PVA), cellulose, COOH—(CH₂)ₙ—COOH (e.g. citric acid, succinic acid, glutaric acid) NH₂—(CH₂)ₙ—NH₂ (e.g. 1,4-butane diamine, 1,6-hexane diamine, cystamine, SH—(CH₂)ₙ—COOH (e.g. mercapto-acetic acid, mercapto-propanoic acid, mercapto dodecanoic acid), Iminothiolane hydrochloride, dicarboxylic acid moieties, PEG moieties (e.g. NH₂-PEG-COOH, NH₂-PEG-NH₂, COOH-PEG-COOH, NH₂-PEG-SH, SH-PEG-COOH), N-hydroxysuccinimide ester (e.g. N-Hydroxysuccinimide ester-poly(ethylene glycol)-b-poly(ε-caprolactone), N-Hydroxysuccinimide ester-poly (ethylene glycol)-b-poly(D,L lactide)), sulfo-N-hydroxysuccinimide ester, (PEGylated bis(sulfosuccinimidyl)suberate)N-γ-maleimidobutyryl-oxysuccinimide ester (GMBS) (e.g. 0-[N-(3-Maleimidopropionyl) amino ethyl]-0′-[3-(N-succinimidyloxy)-3-4-maleimidobutyrate sodium salt, Sulfo-KMUS (oxopropyl] heptacosa ethylene glycol), N-γ-maleimidobutyryl-oxysulfosuccinimide ester (sulfo-GMBS) (e.g. Sulfo-N-succinimidyl N—(κ-maleimido undecanoyloxy) sulfosuccinimide ester) or a combination thereof are exemplary spacers suitable for use in any of the aspects or embodiments described herein.

### Example 10

Functionalization of Glass Beads with (3-Aminopropyl)Triethoxysilane

1 gram glass beads of 2 millimeter diameter were first washed with 10 milliter water containing 1.6 gram of sodium hydroxide, over rocker shaker for 10 minutes, followed by washing with distilled water and drying in oven at 100° C. for an hour. 1 gram of dried glass beads were then added to 10 milliliter piranha solution and heated at 80-100° C. for 2 hours. The reaction mixture was then allowed to cool to room temperature. The glass beads were filtered and washed with 5 milliliter water and 5 milliliter ethanol and dried in oven at 100° C. for an hour. For functionalization, 1 gram of above dried glass beads were immersed in solution of 0.5 milliliter (3-aminopropyl)triethoxysilane in 5 milliliter of toluene and shaken for 2 hours over rocker shaker at room temperature. After 2 hours, glass beads were filtered and washed with 5 milliliter of toluene followed by 5 milliliter of acetone and finally dried in oven at 110° C. for 8 hours. Functionalized glass beads were stored in ethanol at 4° C.

The (3-aminopropyl)silane glass beads were transparent in colour. The Fourier transform infrared spectroscopy in the spectral region from 1389 to 771 cm⁻¹ showed the presence of —N—H— bend and —Si—O—Si— (silane functionality) confirming the silanization on glass.

To those skilled in the art, it would be apparent that the reaction can be carried out using different glass substrate geometries selected from glass beads of different diameters, glass capillary tubes of different internal diameters, and glass cover slips.

### Example 11

Functionalization of Glass Beads with (3-glycidyloxypropyl)trimethoxysilane

1 gram glass beads 2 millimeter in diameter were first washed with 10 milliliter water containing 1.6 gram sodium hydroxide over rocker shaker for 10 minutes, followed by washing with distilled water and drying in oven at 100° C. for an hour. The beads were then added to 10 milliliter of piranha solution and heated at 80-100° C. for 2 hours. The reaction mass was allowed to cool to room temperature, then glass beads were filtered and washed with water until the wash water was no more acidic. The beads were washed with 5 milliliter of ethanol and dried in oven at 100° C. for an hour. For functionalization, glass beads were immersed in solution of 0.5 milliliter (3-glycidyloxypropyl)trimethoxysilane in 5 milliliter toluene and shaken for 2 hours over rocker shaker at room temperature. After 2 hours, glass beads were filtered and washed with 5 milliliter of toluene followed by 5 milliliter of acetone and finally dried in oven at 110° C. for 8 hours. Functionalized glass beads were stored in ethanol at 4° C.

The (3-glycidyloxypropyl)silane glass beads were transparent in colour. The Fourier transform spectroscopy in the spectral region 866 cm⁻¹ showed the presence of oxirane confirming the silanization on glass.

Other reagents for functionalization of glass beads were selected from (3-glycidyloxypropyl) trimethoxysilane (GPTMS) and (3-aminopropyl)trimethoxysilane (APTMS), (3-glycidyloxypropyl)triethoxysilane (GPTES), (3-mercaptopropyl)trimethoxysilane (MPTMS), (3-mercaptopropyl)triethoxysilane (MPTES), 3-(trimethoxysilyl)propyl methacrylate. The reaction can be carried out in solvents selected from ethanol, acetic acid, acetone, cyclohexane, hexane, octane, cyclooctane or in mixture of solvents. The reaction time was varied by carrying out reactions at temperature between 25-90° C. The degree of functionalization was varied by varying the amount of functionalization agent and the degree of functionalization was quantified using colorimetric Orange II dye method.

Functionalization was carried out on the surfaces of glass in the form of cover slips, glass slides and on the interior surfaces of glass capillaries by adapting above method.

### Example 12

Linking of Glutathione Spacer with (3-aminopropyl)silane Functionalized Glass Beads

40 microgram glutathione and 100 milligram glass beads of example 10 were added to the solution of 76 microgram N-(3-dimethylaminopropyl)-N′-ethylcarbodiimide hydrochloride and 46 microgram N-hydroxysuccinimide in 0.2 milliliter phosphate buffered saline pH 7.4 and vortexed for 3 hours. The glass beads were filtered, washed with 1 milliliter distilled water three times and dried in oven at 60° C. for an hour. The product was stored in distilled water.

The glutathione functionalized (3-aminopropyl)silane glass beads were transparent in colour. The Fourier transform infrared spectroscopy in the spectral region from 1720 to 1400 cm⁻¹ showed the presence of amide group and peak at 3346 cm⁻¹ showed the presence of carboxylic acid groups of glutathione, thereby confirmed the linking of glutathione spacer on (3-aminopropyl)silane functionalized glass beads.

### Example 13

Linking of Succinic Acid Spacer with (3-aminopropyl)silane Functionalized Glass Beads

100 milligram (3-aminopropyl)silane functionalized glass beads of example 10 were added to a solution of 1 milligram succinic anhydride and 50 microliter N,N-diisopropylethylamine in 1.5 milliliter dimethylformamide. The reaction mixture was vortexed at 50 rotations per minute for 3 hours. The reaction mixture was filtered on a sintered glass funnel and washed with 10 milliliter distilled water three times to yield succinic acid linked functionalized glass bead product. Finally the glass beads were dried in oven at 60° C. for an hour and stored in distilled water.

Functionalized (3-aminopropyl)silane glass beads linked to succinic acid spacer were transparent and colorless. The Fourier transform infrared spectroscopy in the spectral region 1700 cm⁻¹ showed the presence of carboxylate groups (—COO⁻) confirming the linkage of succinic acid on (3-aminopropyl)silane functionalized glass beads.

A molecule for linking to functionalized glass beads as a spacer can be selected from succinic anhydride, cysteine, glutaraldehyde, aspartic acid, mercapto-acetic acid, mercapto-propanoic acid, iminothiolane hydrochloride, dicarboxylic acid moieties, PEG moieties (e.g. NH₂-PEG-COOH, NH₂-PEG-NH₂, COOH-PEG-COOH, NH₂-PEG-SH, SH-PEG-COOH), poly(methacrlyic acid) (PMA), poly(oligo(ethylene glycol) methacrylate, poly(oligo(ethylene glycol)diglycidyl ether, poly(N-isopropyl acrylamide) (PNIPAM), polyimines, poly(amidoamine) (PAMAM), poly(acrylic acid) (PAA), poly(ethylene-co-acrylic acid), poly(lactic acid) (PLA), 2-aminoethoxy acetic acid, NH₂—(CH₂)ₙ—COOH (e.g. 6-amino hexanoic acid, amino-propionic acid, amino-butanoic acid, amino-dodecanoic acid), molecules with hydroxy groups (eg. Polyvinyl alcohol (PVA), cellulose), COOH—(CH₂)ₙ—COOH (e.g. citric acid, succinic acid, glutaric acid), NH₂—(CH₂)ₙ—NH₂ (e.g. 1,4-butane diamine, 1,6-hexane diamine, cystamine), SH—(CH₂)ₙ—COOH (e.g. mercapto-acetic acid, mercapto-propanoic acid, mercapto-dodecanoic acid, N-hydroxy succinimide ester (e.g. N—Hydroxysuccinimide ester-poly(ethylene glycol)-b-poly(ε-caprolactone), N—Hydroxysuccinimide ester-poly (ethylene glycol)-b-poly(D,L lactide) etc), sulfo-N-hydroxysuccinimide ester (BS(PEG)9 (PEGylated bis (sulfosuccinimidyl)suberate)N-γ-maleimidobutyryl-oxysuccinimide ester (GMBS) (e.g. O—[N-(3-Maleimidopropionyl) aminoethyl]-O′-[3-(N-succinimidyloxy)-3-oxopropyl] heptacosaethylene glycol), N-γ-maleimidobutyryl-oxysulfosuccinimide ester (sulfo-GMBS) (e.g. Sulfo-N-succinimidyl 4-maleimidobutyrate sodium salt, Sulfo-KMUS (N-(κ-maleimidoundecanoyloxy) sulfosuccinimide ester), peptides or a combination thereof.

### Example 14

Crosslinking of Glutathione Functionalized Iron Oxide Particles

10 milligram iron oxide nanoparticles of example 2 were dispersed in 1 milliliter distilled water. 383 microgram N-(3-dimethylaminopropyl)-N′-ethylcarbodiimide hydrochloride and 230 microgram N-hydroxysuccinimide in 2 milliliter distilled water was added to the above mixture and vortexed at room temperature for 4 hours. The reaction mixture was then placed over magnet and supernatant was removed. The residue was washed with 1 milliliter of distilled water. The particles obtained were dried in oven at 60° C. and stored at 4° C.

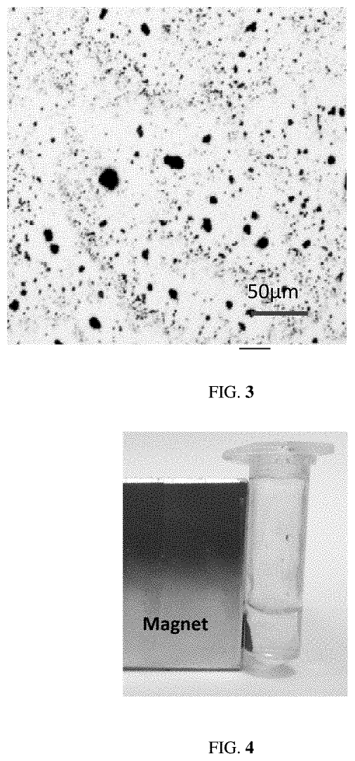

The microscopy image of crosslinked iron oxide particles showed agglomerates (see FIG. 3, 4). The crosslinked iron oxide particles were brown in colour and retained paramagnetic properties The particle size ranged from 0.2 micron to 2.0 micron (see FIG. 3). The spectral peak in Fourier transform infrared spectroscopy at 1628 cm⁻¹ showed the amide functionality and 1041 cm⁻¹ denoted the disulphide functionality, thereby confirming crosslinking of functionalized iron oxide nanoparticles.

### Example 15

Crosslinking of Functionalized Iron Oxide Particles

100 milligram iron oxide nanoparticles of example 6 and 100 milligram iron oxide nanoparticles of example 7 were dispersed in 50 milliliter carbonate buffer pH 9.2, followed by vortexing for 12 hours. The reaction flask was placed over a magnet and supernatant was removed. 10 milliliter fresh distilled water was added to the flask to disperse the particles and again placed over a magnet to remove supernatant. The purification was done by washing with 10 milliliter of distilled water and ethanol alternatively until the washings became colourless. The particles obtained were dried in an oven at 60° C. and stored in dry state at 4° C.

The crosslinked iron oxide particles were brown in colour and retained paramagnetic properties. The spectral peaks in Fourier transform infrared spectroscopy at 3406 cm⁻¹ and 1626 cm⁻¹ showed the presence of —N—H— functionalities from (3-aminopropyl)silane and (3-glycidyloxypropyl)silane confirming crosslinking, moreover the peak at 994 cm⁻¹ further confirmed silane moieties present on crosslinked iron oxide particles.

### Example 16

Crosslinking of (3-aminopropyl)silane Functionalized Glass Beads Using Glutaraldehyde

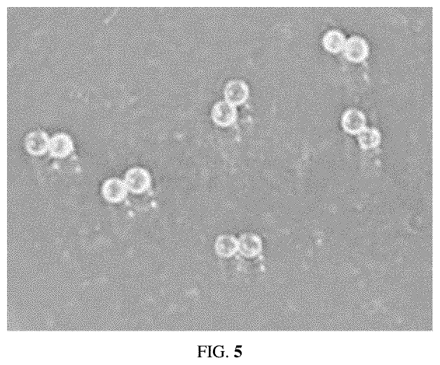

100 milligram of glass beads of example 10 were vortexed with 10 milligram glutaraldehyde in 0.2 milliliter distilled water pH 5.0 for 4 hours. The beads were washed with distilled water and added to 0.2 milliliter distilled water pH 5.0 containing 100 milligram glass beads of example 10, followed by mixing for 4 hours. The crosslinked beads formed were washed with phosphate buffered saline at pH 7.4, dried and stored at 4° C. (FIG. 5). The glass beads recovered were transparent.

### Example 17

Crosslinking of (3-aminopropyl)silane Functionalized Glass Beads Linked to Glutathione Functionalized Iron Oxide Nanoparticles with (3-aminopropyl)silane Functionalized Glass Beads Using Glutaraldehyde

100 milligram glutathione functionalized iron oxide nanoparticles linked to (3-aminopropyl)silane functionalized glass beads of example 21 (see later) were treated with 1 milligram glutaraldehyde in 1 milliliter distilled water pH 5.0 for 4 hours. Thereafter the buffer was removed and glutaraldehyde linked glass beads were washed with 1 milliliter distilled water three times. 100 milligram glutaraldehyde linked glass beads and 100 milligram (3-aminopropyl)silane functionalized glass beads with 2 millimeter diameter of example 10 was vortexed in distilled water pH 5.0 for 4 hours. After 4 hours, the supernatant was decanted and the product was washed with 1 milliliter distilled water twice. The final product was dried in oven at 60° C. and stored in distilled water.

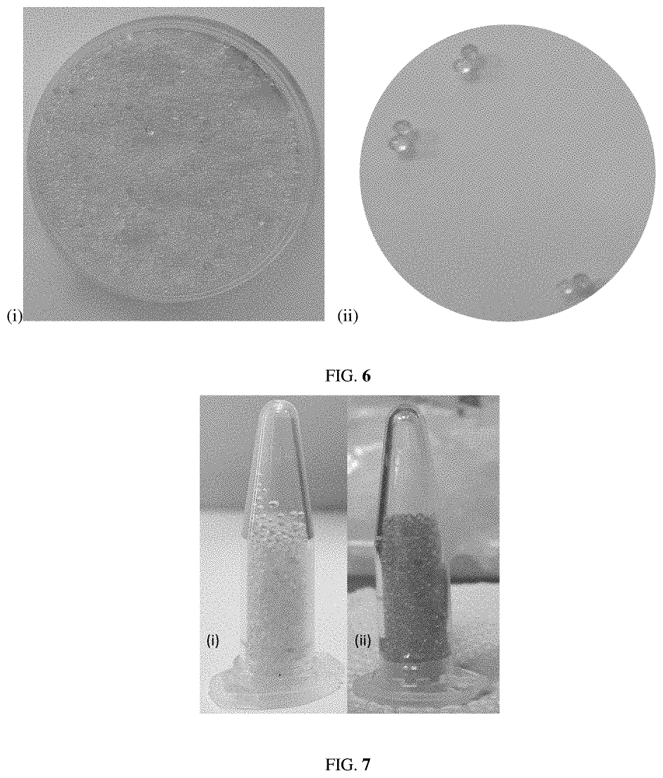

FIG. 6 (ii) shows the crosslinked (3-aminopropyl)silane functionalized glass beads linked to glutathione functionalized iron oxide nanoparticles with (3-aminopropyl)silane functionalized glass beads using glutaraldehyde. These glass beads crosslinked using glutaraldehyde were brown in colour and paramagnetic in nature.

### Example 18

Crosslinking of (3-mercaptopropyl)silane Functionalized Glass Beads with Iron Oxide Nanoparticles Followed by Functionalization with Citric Acid

100 milligram (3-mercaptopropyl)silane functionalized glass beads of 2 millimeter diameter were sonicated with 1 milligram iron oxide nanoparticles of example 1 in a mixture of 3 milliliter distilled water and 1 milliliter methanol for 1 hour. The iron oxide nanoparticles in supernatant were decanted out and fresh 1 milliliter distilled water was added to the reaction mixture. The reaction mixture was vortexed and the supernatant was removed. This was continued until the supernatant was colourless. 1 milligram citric acid was dissolved in h 3 milliliter of distilled water and added to above mixture and the reaction mixture was stirred for 1 hour at 80° C. The product was washed with 1 milliliter distilled water thrice, followed by drying in oven at 60° C. and stored in distilled water at 4° C.

The physical mixture of (3-mercaptopropyl)silane functionalized glass beads with iron oxide nanoparticles washed with distilled water, showed no physical adsorption of iron oxide on (3-mercaptopropyl)silane functionalized glass beads. The (3-mercaptopropyl)silane functionalized glass beads crosslinked with iron oxide nanoparticles followed by functionalization with citric acid were brown in colour and paramagnetic. No leaching of iron oxide nanoparticles was observed in water/organic solvent over 6 months.

### Example 19

Crosslinking of (3-aminopropyl)silane Functionalized Glass Beads with Iron Oxide Nanoparticles Using Iminothiolane Hydrochloride Followed by Functionalization with Glutathione

100 milligram glass beads of example 10 were treated with 1 milligram iron oxide nanoparticles of example 1 in the presence of 40 microgram iminothiolane hydrochloride dissolved in 0.5 milliliter phosphate buffered saline pH 7.2 for 4 hours. The supernatant from the reaction mixture was removed and the beads were washed with 5 milliliter of distilled water. These beads were added to solution containing 1 milligram glutathione in 3 milliliter of distilled water and 1 milliliter of methanol. The reaction mixture was stirred for 1 hour and the reaction product was dried in oven at 60° C. and stored in distilled water at 4° C.

The physical mixture of iron oxide nanoparticles and (3-aminopropyl)silane functionalized glass beads washed with distilled water, showed no physical adsorption of iron oxide nanoparticles on (3-aminopropyl)silane functionalized glass beads. The (3-aminopropyl)silane functionalized glass beads crosslinked with iron oxide nanoparticles using iminothiolane hydrochloride followed by functionalization with glutathione were brown in colour and paramagnetic. No leaching of iron oxide nanoparticles was observed in water/organic solvent over 6 months.

### Example 20

Crosslinking of Iron Oxide Nanoparticles with Succinic Acid Functionalized (3-aminopropyl)silane Glass Beads Followed by Functionalization with Glutathione

1 milligram iron oxide nanoparticles of example 1 were dispersed in 0.2 milliliter distilled water and 100 milligram of glass beads of example 13 were added. The reaction mixture was vortexed for 12 hours and the supernatant was discarded. Fresh 1 milliliter distilled water was added to the residue and it was sonicated. The supernatant was discarded and the product was isolated. This was added to the solution containing 1 milligram glutathione in 3 milliliter of distilled water and 1 milliliter of methanol. The reaction mixture was stirred for 1 hour and the reaction product was dried in oven at 60° C. and stored in distilled water at 4° C. This product was brown in colour and no leaching of iron oxide was observed for 6 months.

The physical mixture of samples of examples 1 and 13 when washed with distilled water showed no physical adsorption of iron oxide nanoparticles.

### Example 21

Crosslinking of Glutathione Functionalized Iron Oxide Nanoparticles with (3-aminopropyl)silane Functionalized Glass Beads

1 milligram iron oxide nanoparticles of example 2 were treated with 383 microgram N-(3-dimethylaminopropyl)-N′-ethylcarbodiimide hydrochloride and 230 microgram N-hydroxysuccinimide in 1 milliliter phosphate buffered saline pH 6.0 for 4 hours. The reaction mixture was filtered and washed with distilled water and added to 100 milligram (3-aminopropyl)silane-glass beads of example 10 in 1 milliliter phosphate buffered saline pH 7.2. The reaction mixture was vortexed for 4 hours at room temperature. Thereafter, the supernatant was decanted and 1 milliliter distilled water was added to the reaction flask. The flask was shaken mechanically to remove any free glutathione-iron oxide particles and the supernatant was discarded. The washing procedure was continued until the discarded solvent was colourless. Finally, the reaction product was dried in oven at 60° C. and stored in distilled water at 4° C.

The physical mixture of iron oxide nanoparticles of example 2 with (3-aminopropyl)silane functionalized glass beads and washed with distilled water, showed no physical adsorption of iron oxide on glass beads. The glutathione functionalized iron oxide nanoparticles crosslinked with (3-aminopropyl)silane glass beads were brown in colour and paramagnetic. FIG. 7 show the change in colour after crosslinking glutathione functionalized iron oxide nanoparticles with glass beads of example 10. Similar reaction was carried out on glass cover slip. FIG. 8 (i) shows the microscopy image of crosslinked glutathione functionalized iron oxide nanoparticles with (3-aminopropyl)silane functionalized on glass coverslip.

No leaching of iron oxide nanoparticles was observed in water/organic solvent over 6 months.

To those skilled in the art it would be apparent that the reaction can be carried out in distilled water at pH ranging from 4 to 10 and also in a solvent selected from. (2-(N-morpholino) ethane sulfonic acid, phosphate buffered saline, (4-(2-hydroxyethyl)-1-piperazineethanesulfonic acid buffer or dimethyl formamide, dimethyl sulfoxide.

### Example 22

Crosslinking of Citric Acid Functionalized Iron Oxide Nanoparticles with (3-aminopropyl)silane Functionalized Glass Beads

100 milligram glass beads of example 10, and iron oxide nanoparticles of example 5 were dispersed in 1 milliliter phosphate buffered saline of pH 7.2. A solution of 383 microgram N-(3-Dimethylaminopropyl)-N′-ethyl carbodiimide hydrochloride and 230 microgram N-hydroxysuccinimide in 1 milliliter phosphate buffered saline pH 7.2 was added to the solution. The reaction mixture was vortexed for 4 hours at room temperature. The product was purified by discarding the supernatant and washing glass beads with 100 milliliter phosphate buffered saline pH 7.2 thrice. The product was stored in dried state at 4° C.

The product was brown in colour and paramagnetic. No leaching of iron oxide nanoparticles was observed in water/organic solvent over 6 months.

The spectral peak in Fourier transform spectroscopy at 1379 cm-1 confirmed the crosslinking of citric acid functionalized iron oxide nanoparticle with (3-aminopropyl)silane functionalized glass beads.

The physical mixture of iron oxide nanoparticles of example 5 and glass beads of example 10 washed with distilled water showed no physical adsorption of iron oxide on glass beads.

### Example 23

Crosslinking of (3-aminopropyl)silane Functionalized Glass Beads with Iron Oxide Nanoparticles Functionalized with Citric Acid and Linked with 12-Aminododecanoic Acid

100 milligram glass beads of example 10, were added to iron oxide nanoparticles of example 8, and dispersed in 0.2 milliliter phosphate buffered saline pH 7.2. A mixture of 383 microgram N-(3-Dimethylaminopropyl)-N′-ethyl carbodiimide hydrochloride and 230 microgram N-hydroxysuccinimide in 1 milliliter phosphate buffered saline pH 7.2 was added to the solution. The rection mixture was vortexed for 4 hours at room temperature. The product was purified by decanting the supernatant and washing glass beads with 10 milliliter phosphate buffered saline pH 7.2 thrice. The product was stored in dried state at 4° C. The product was brown and paramagnetic.

No leaching of iron oxide nanoparticles was observed in water/organic solvent over 6 months.

The Fourier transform infrared spectrum with spectral region at around 883 cm-1 denotes —C—H— stretch which confirms the linkage of 12-aminododecanoic acid to citric acid functionalized iron oxide nanoparticles.

The physical mixture of iron oxide nanoparticles of example 8 and glass beads of example 10 washed with distilled water, showed no physical adsorption of iron oxide on glass beads.

### Example 24

Linking of Glutathione Functionalized Iron Oxide Nanoparticle with Anti-Epithelial Cell Adhesion Molecule Antibody

10 milligram iron oxide nanoparticles of example 2 were dispersed in 2-(N-morpholino) ethanesulfonic acid buffer pH 6.0 (1 milliliter). A solution of 383 micrograms N-(3-imethylaminopropyl)-N′-ethylcarbodiimide hydrochloride and 230 micrograms N hydroxysuccinimide in 1 milliliter 2-(N-morpholino)ethanesulfonic acid buffer pH 6.0 was added to the above mixture and incubated for 1 hour. These nanoparticles were separated using magnet and washed with 1 milliter phosphate buffered saline at pH 7.2 thrice. These nanoparticles were re-dispersed in 2 milliliter phosphate buffered saline pH 7.2 and 1 microgram of anti-epithelial cell adhesion molecule antibody was added to it. The reaction mixture was vortexed for 4 hours at 4° C., followed by their separation using a magnet and washing with 1 milliliter phosphate buffered saline pH 7.2 thrice. Finally, the product was stored in 200 microliter phosphate buffered saline pH 7.2 at 4° C. The product was brown in colour and paramagnetic in nature.

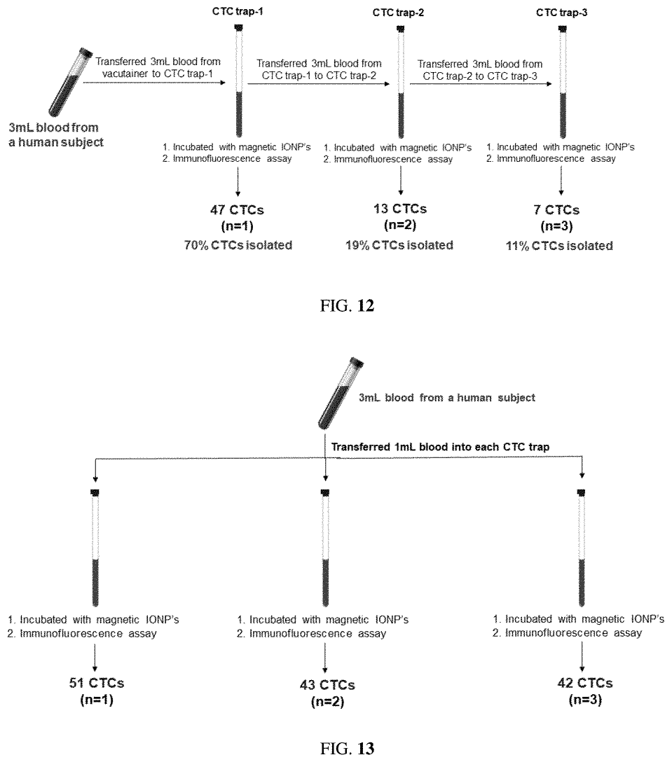

The linkage of anti-epithelial cell adhesion molecule antibody to glutathione functionalized iron oxide nanoparticles was confirmed by the CTC capturing assay (see example 59). FIG. 12,13,16 showed the capture of CTCs using anti-epithelial cell adhesion molecule antibody linked glutathione functionalized iron oxide nanoparticles. CTCs were not captured in absence of anti-epithelial cell adhesion molecule antibody linking to glutathione functionalized iron oxide nanoparticles.

### Example 25

Linking of Glutathione Functionalized Iron Oxide Nanoparticles with Transferrin

10 milligram iron oxide nanoparticles of example 2 were dispersed in 1 milliliter 2-(N-morpholino)ethanesulfonic acid buffer pH 6.0. 383 micrograms N-(3-Dimethylaminopropyl)-N′-ethylcarbodiimide hydrochloride and 230 micrograms N-hydroxysuccinimide dissolved in 1 milliliter 2-(N-morpholino)ethanesulfonic acid buffer pH 6.0 was added to the above mixture and incubated for 1 hour. These nanoparticles were separated using magnet and washed with 1 milliliter phosphate buffered saline pH 7.2 thrice. These nanoparticles were dispersed in 2 milliliter phosphate buffered saline pH 7.2 and 1 microgram transferrin was added to it. The reaction mixture was vortexed for 4 hours at 4° C. and the product was separated using magnet and washed with 1 milliliter phosphate buffered saline pH 7.2 thrice. Finally the product was stored in 0.2 milliliter phosphate buffered saline pH 7.2 at 4° C.

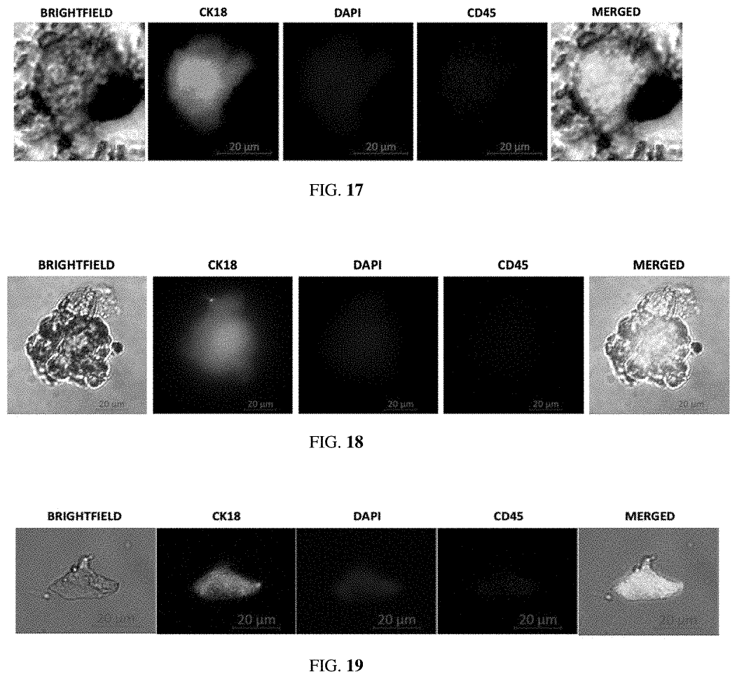

The product was brown in colour and paramagnetic in nature. The linkage of transferrin was confirmed by the CTC capturing assay (see example 59). FIG. 17 shows the capture of CTCs using transferrin functionalized iron oxide nanoparticles. CTCs were not captured in absence of transferrin on to glutathione functionalized iron oxide nanoparticles.

### Example 26

Linking of Glutathione Functionalized Iron Oxide Nanoparticles with Bovine Serum Albumin

10 milligram iron oxide nanoparticles of example 2 were dispersed in 1 milliliter 2-(N-morpholino)ethanesulfonic acid buffer pH 6.0. A solution of 383 micrograms N-(3-dimethylaminopropyl)-N′-ethylcarbodiimide hydrochloride and 230 micrograms N-hydroxysuccinimide in 1 milliliter 2-(N-morpholino)ethanesulfonic acid buffer pH 6.0 was added to the above mixture, followed by incubation for 1 hour. The nanoparticles were separated using magnet and washed with 1 milliter phosphate buffered saline pH 7.2 thrice. These were re-dispersed in 2 milliliter phosphate buffered saline pH 7.2 and 1 microgram bovine serum albumin was added to it. The reaction mixture was vortexed for 4 hours at 4° C. and the reaction product was isolated using magnet and washed with 1 milliliter phosphate buffered saline pH 7.2 thrice. Finally the product was stored in 0.2 milliliter phosphate buffered saline pH 7.2 at 4° C.

The glutathione functionalized iron oxide nanoparticles linked with bovine serum albumin were brown in colour and paramagnetic in nature. The linkage of bovine serum albumin was confirmed by drug recovery assay using metformin hydrochloride and vancomycin hydrochloride (see example 54). Drug recovery was not observed in the case of glutathione functionalized iron oxide nanoparticles sans bovine serum albumin.

### Example 27

Linking of Glutathione Functionalized Iron Oxide Nanoparticle with N-Acetyl Glucosamine

10 milligram iron oxide nanoparticles of example 2 and 10 milligram N-acetyl glucosamine were dispersed in 1 milliliter distilled water. 2 milligram N-(3-Dimethylaminopropyl)-N′-ethylcarbodiimide hydrochloride, and 50 micrograms 4-dimethylaminopyridine in 1 milliliter distilled water was added to the above solution. The reaction mixture was vortexed for 3 hours and glutathione functionalized iron oxide nanoparticles linked to N-acetyl glucosamine were isolated using magnet and washed with 5 milliliter of phosphate buffered saline pH 7.2 thrice. These nanoparticles were stored in dried state at 4° C.

The product was brown in colour and paramagnetic. The spectral peak in Fourier transform infrared spectrum at 1000 cm⁻¹ depicted the presence of C—O stretch bend and the peak at 1100 cm⁻¹ confirm the presence of ether linkage of N-acetyl glucosamine. The ester peak at 1500 cm⁻¹ confirmed the presence of ester linkage of glutathione functionalized iron oxide nanoparticle with N-acetyl glucosamine. The linkage of N-acetyl glucosamine was confirmed by protein recovery using bovine serum albumin (see example 55). Protein recovery was not observed in the case of glutathione functionalized iron oxide nanoparticles sans N-acetyl glucosamine.

### Example 28

Linking of (3-glycidyloxypropyl)silane Functionalized Glass Beads with Anti-Epithelial Cell Adhesion Molecule Antibody.

100 milligrams glass beads of example 11, were treated with 1 microgram of anti-epithelial cell adhesion molecule antibody in 200 microliter phosphate buffered saline pH 7.2 for 4 hours at 4° C. The glass beads were washed with 1 milliliter phosphate buffered saline pH 7.2 thrice and finally stored in 0.2 milliliter phosphate buffered saline pH 7.2 at 4° C.

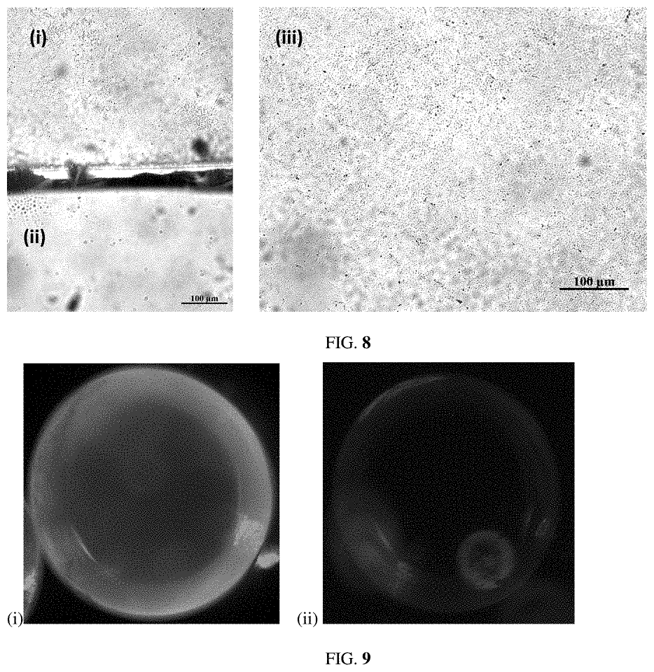

The product was transparent in nature. The linking of anti-epithelial cell adhesion molecule antibody to (3-glycidyloxypropyl)silane functionalized glass bead was confirmed by fluorescein isothiocyanate. The fluorescein isothiocyanate intensity of anti-epithelial cell adhesion molecule antibody linked to glass beads was 987.372 which was four times higher than that for (3-glycidyloxypropyl)silane functionalized glass bead sans anti-epithelial cell adhesion molecule antibody (241.64) (see FIG. 9). The increase in intensity confirmed the linking of anti-epithelial cell adhesion molecule antibody to (3-glycidyloxypropyl)silane functionalized glass bead (FIG. 9).

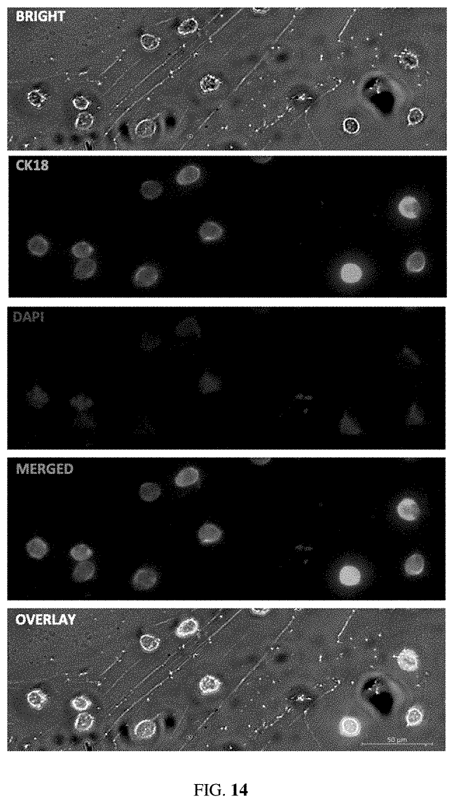

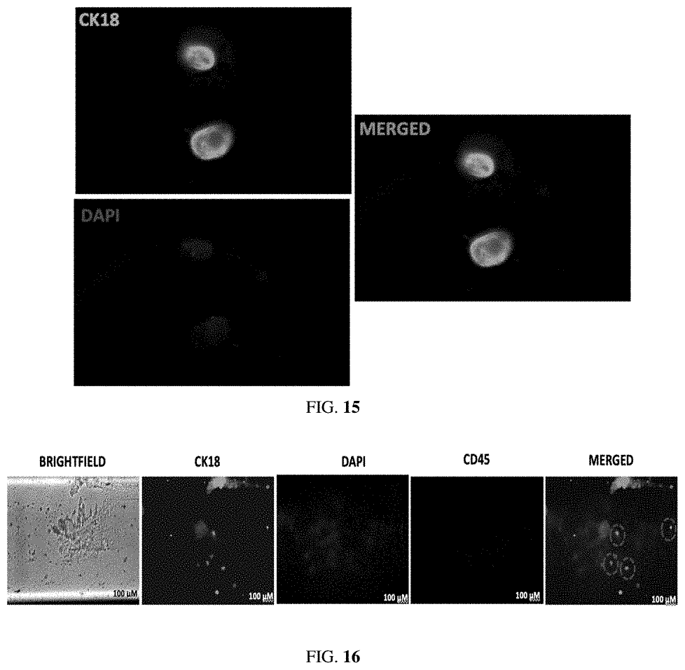

The linkage of (3-glycidyloxypropyl)silane functionalized glass beads to anti-epithelial cell adhesion molecule antibody was confirmed by the CTC and HCT-116 capturing assay (see example 58, 59). FIG. 14,15 shows the capture of HCT-116 cells using anti-epithelial cell adhesion molecule antibody linked to (3-glycidyloxypropyl)silane functionalized using glass cover slip and glass beads. FIG. 18 shows the capture of circulating tumor cell cluster using anti-epithelial cell adhesion molecule antibody linked to (3-glycidyloxypropyl)silane functionalized glass cover slip. HCT-116 cells and CTCs/cluster were not captured when the anti-epithelial cell adhesion molecule antibody was absent on (3-glycidyloxypropyl)silane functionalized glass cover slip and on beads.

It would be apparent to the persons skilled in the art that ligands which can bind to the target transferrin, bovine serum albumin, N-acetyl glucosamine and other active biomolecules can also be linked to glass beads by varying the conditions of linking.

### Example 29

Linking of (3-aminopropyl)silane Functionalized Glass Beads Linked with Glutathione Spacer to Anti-Epithelial Cell Adhesion Molecule Antibody

100 milligram glass beads of example 12 were added to 80 microgram N-(3-dimethylaminopropyl)-N′-ethylcarbodiimide hydrochloride and 50 microgram N-hydroxysuccinimide in 200 microliter 2-(N-morpholino)ethanesulfonic acid buffer pH 6.0 and were vortexed for an hour, followed by washing with 3 milliliters of phosphate buffered saline pH 7.2, three times. 200 microliter phosphate buffered saline pH 7.2, containing 1 microgram of anti-epithelial cell adhesion molecule antibody was added to the glass beads. The reaction mixture was stirred at 4° C. for 4 hours. The glass beads were then washed with 1 milliliter phosphate buffered saline pH 7.4 thrice and stored in 0.2 milliliter phosphate buffered saline pH 7.4 at 4° C.

The final product was transparent. The linking of anti-epithelial cell adhesion molecule antibody was confirmed by fluorescein isothiocyanate dye. The fluorescein isothiocyanate intensity of anti-epithelial cell adhesion molecule antibody was 856.23 vis a vis that for glass beads anti-epithelial cell adhesion molecule antibody (263.43).

### Example 30

Linking of (3-aminopropyl)silane Functionalized Glass Beads Linked with Glutathione Spacer to Transferrin

100 milligram glass beads, of example 12 were added to 80 microgram N-(3-dimethylaminopropyl)-N′-ethylcarbodiimide hydrochloride and 50 microgram N-hydroxysuccinimide in 200 microliter 2-(N-morpholino)ethanesulfonic acid buffer pH 6.0 and was vortexed for an hour, followed by washing with phosphate buffered saline pH 7.2 thrice. Finally, 200 microliter phosphate buffered saline pH 7.2 containing 1 microgram transferrin was added to the glass beads. The reaction mixture was stirred at 4° C. for 4 hours. The glass beads were washed with 1 milliliter phosphate buffered saline pH 7.4 thrice and stored in 0.2 milliliter phosphate buffered saline pH 7.4 at 4° C.

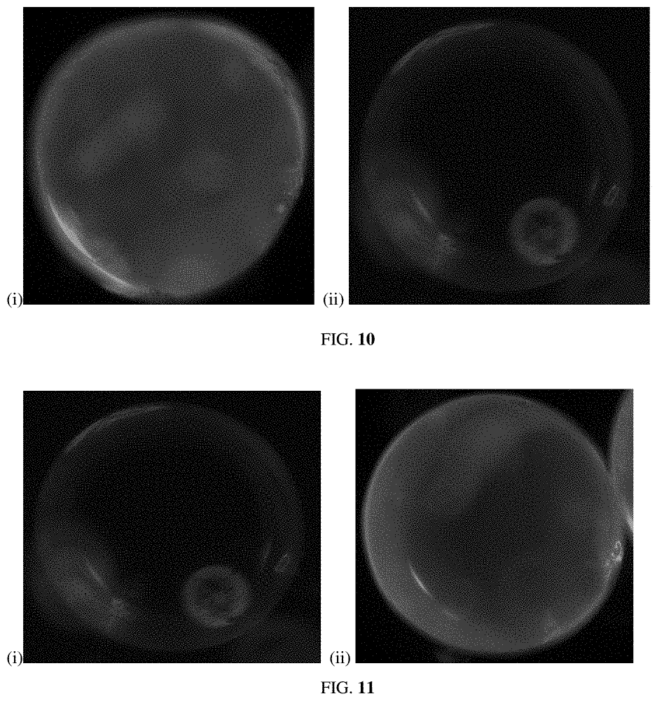

The final product was transparent. The linking of transferrin was confirmed by using fluorescein isothiocyanate. The fluorescein isothiocyanate intensity of transferrin linked to glass beads was 569.745 which was higher than in the case when transferrin was absent (241.643) (FIG. 10).

The linkage of transferrin with (3-aminopropyl)silane functionalized glass beads linked with glutathione spacer was confirmed by the CTC capturing assay. (see example 59). FIG. 19 shows the capture of CTCs using (3-aminopropyl)silane functionalized glass beads linked with glutathione to transferrin. CTCs were not captured when transferrin was not linked to (3-aminopropyl)silane functionalized glass beads.

### Example 31

Linking of (3-aminopropyl)silane Functionalized Glass Beads Linked with Glutathione Spacer to Bovine Serum Albumin

100 milligrams glass beads of example 12 were added to 80 micrograms N-(3-dimethyl aminopropyl) —N′-ethylcarbodiimide hydrochloride and 50 micrograms N-hydroxysuccinimide in 200 microliter 2-(N-morpholino)ethanesulfonic acid buffer pH 6.0 and was vortexed for an hour, followed by washing with phosphate buffered saline pH 7.2 thrice. 200 microliter phosphate buffered saline pH 7.2 containing 1 microgram bovine serum albumin was added to the glass beads. The reaction mixture was shaken over a rocker shaker at 4° C. for 4 hours. The glass beads were then washed with 1 milliliter phosphate buffered saline pH 7.4 thrice and stored in 0.2 milliliter phosphate buffered saline pH 7.4 at 4° C.

The final product was transparent. The linking of bovine serum albumin was confirmed by fluorescein isothiocyanate. The fluorescein isothiocyanate intensity of bovine serum albumin linked to glass beads was 628.804 which was higher than that for glass beads sans bovine serum albumin (241.643). (FIG. 11). The linkage of bovine serum albumin was further confirmed by drug recovery assay using metformin hydrochloride and vancomycin hydrochloride (see example 54). Drug recovery was not observed in absence of bovine serum albumin.

### Example 32

Linking of (3-aminopropyl)silane functionalized Glass Beads Linked with Glutathione Spacer to N-acetyl Glucosamine

100 milligrams of glass beads of example 12 and 80 microgram N-acetyl glucosamine were added to 200 microgram N-(3-dimethylaminopropyl)-N′-ethylcarbodiimide hydrochloride and 10 microgram 4-dimethylaminopyridine in 1 milliliter distilled water and was vortexed for 3 hours. Thereafter, glass beads were washed with 1 milliliter distilled water thrice and dried in oven at 60° C. and stored at 4° C.

The final product was transparent in nature. The spectral peaks in Fourier transform infrared spectroscopy at 1500 cm⁻¹ confirmed the presence of ester group, thereby confirming the linking of N-acetyl glucosamine with (3-aminopropyl)silane functionalized glass beads linked with glutathione spacer.

The protein recovery was estimated as described in example 55. Protein recovery was not observed in the case of glass beads sans N-acetyl glucosamine.

### Example 33

Linking of Glutathione Functionalized Crosslinked Iron Oxide Particles to Anti-Epithelial Cell Adhesion Molecule Antibody

5 milligrams crosslinked iron oxide particles of example 14 were dispersed in 1 milliliter 2-(N-morpholino)ethane sulfonic acid buffer pH 6.0. 383 microgram N-(3-dimethylaminopropyl)-N′-ethylcarbodiimide hydrochloride and 230 microgram N-hydroxysuccinimide in 1 milliliter 2-(N-morpholino) ethane sulfonic acid buffer 6.0 were added to the above mixture followed by vortexing for 1 hour. The particles were isolated using magnet and washed with phosphate buffered saline pH 7.2 thrice. The particles were re-dispersed in 1 milliliter phosphate buffered saline pH 7.2 and 1 microgram anti-epithelial cell adhesion molecule antibody was added to the reaction mixture, and vortexed for 4 hours at 4° C. The reaction product was then separated using magnet and washed with 1 milliliter phosphate buffered saline pH 7.2 three times. The final product was stored in 0.2 milliliter phosphate buffered saline pH 7.2 at 4° C.

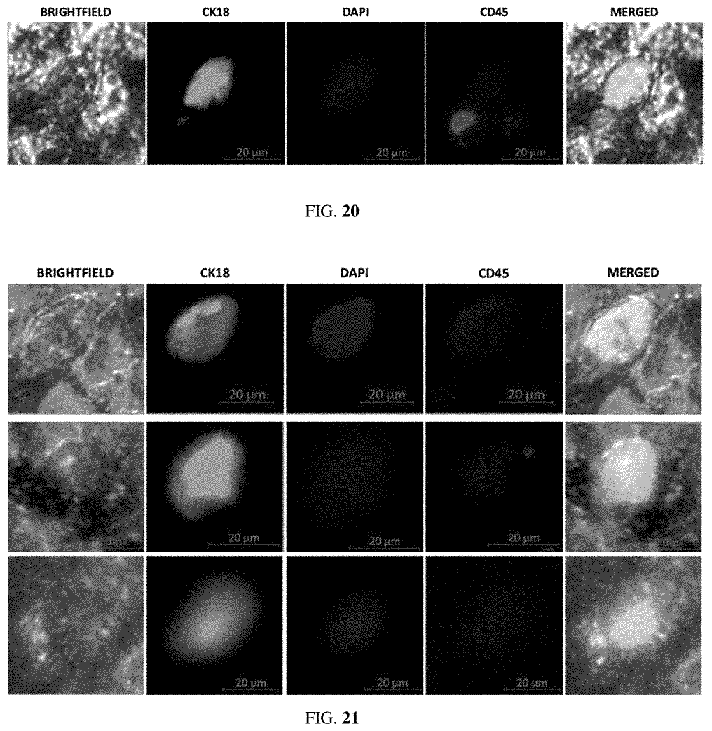

The final product was brown in colour and paramagnetic in nature. The linkage of anti-epithelial cell adhesion molecule antibody to glutathione functionalized crosslinked iron oxide nanoparticles was confirmed by the CTC capturing assay (see example 59). FIG. 20 shows the capture of CTCs using anti-epithelial cell adhesion molecule antibody linked glutathione functionalized crosslinked iron oxide particles. CTCs were not captured in absence of anti-epithelial cell adhesion molecule antibody.

### Example 34

Linking of Glutathione Functionalized Crosslinked Iron Oxide Particles Linked to Amine Terminated Poly(Amidoamine) Dendrimer Spacer to Anti-Epithelial Cell Adhesion Molecule Antibody

20 milligram glutathione functionalized crosslinked iron oxide particles of example 14 and 5 milligram poly(amidoamine) (PAMAM) dendrimer were dispersed in 1 milliliter distilled water. 2 milligram of N-(3-dimethylaminopropyl)-N′-ethylcarbodiimide hydrochloride and 2 milligram N-hydroxysuccinimide in 1 milliliter distilled water was added to the above mixture and vortexed for 3 hours. The reaction mixture was separated and solid residue was washed with 1 milliliter distilled water thrice. The residue was re-dispersed in 1 milliliter distilled water with 100 microgram N-(3-dimethylaminopropyl)-N′-ethylcarbodiimide and 100 microgram N-hydroxysuccinimide in 1 milliliter phosphate buffered saline pH 7.2. 1 microgram of anti-epithelial cell adhesion molecule antibody was added to the above reaction mixture and vortexed for 3 hours at 4° C. The reaction product was isolated using magnet, washed with 1 milliliter phosphate buffered saline pH 7.2 three times. Finally, the reaction product was suspended in 0.2 milliliter phosphate buffered saline pH 7.2 and stored at 4° C.

The final product was brown in colour and paramagnetic in nature.

### Example 35

Linking of Glutathione Functionalized Crosslinked Iron Oxide Particles to Transferrin

10 milligrams crosslinked iron oxide particles of example 14 were dispersed in 1 milliliter 2-(N-morpholino) ethanesulfonic acid buffer pH 6.0. 2 milligram N-(3-dimethylaminopropyl)-N′-ethylcarbodiimide hydrochloride and 2 milligram N-hydroxysuccinimide in 1 milliliter 2-(N-morpholino)ethanesulfonic acid buffer 6.0 was added to above mixture followed by vortexing for 1 hour. Thereafter these particles were separated using magnet and washed with 1 milliliter phosphate buffered saline pH 7.2 three times. The particles were re-dispersed in 1 milliliter phosphate buffered saline pH 7.2 and 1 microgram transferrin was added to the mixture, followed by vortexing for 4 hours. The reaction product was separated using magnet. The solid residue was washed with 1 milliliter phosphate buffered saline buffered pH 7.2 thrice. The product was stored in 0.2 milliliter phosphate buffered saline pH 7.2 at 4° C.

The final product was brown in colour and paramagnetic in nature. The linkage of transferrin was confirmed by the CTC capturing assay (see example 59). FIG. 21 shows the capture of CTCs using glutathione functionalized crosslinked iron oxide particles linked to transferrin. CTCs were not captured when the transferrin was absent on glutathione functionalized crosslinked iron oxide particles.

### Example 36

Linking of Glutathione Functionalized Crosslinked Iron Oxide Particles to Bovine Serum Albumin

10 milligram iron oxide of example 14 were dispersed in 1 milliliter 2-(N-morpholino) ethanesulfonic acid buffer pH 6.0. 2 milligram N-(3-dimethylaminopropyl)-N′-ethylcarbodiimide hydrochloride and 2 milligram N-hydroxysuccinimide in 1 milliliter 2-(N-morpholino)ethanesulfonic acid buffer pH 6.0 was added to the above mixture, followed by vortexing for 1 hour. Thereafter, the solids were separated using magnet and the residue washed with 1 milliliter phosphate buffered saline pH 7.2 thrice. Crosslinked solids were re-dispersed in 2 milliliter phosphate buffered saline pH 7.2 and 1 microgram of bovine serum was added to it. The reaction mixture was vortexed for 4 hours at 4° C. The reaction mixture was separated using a magnet and washed with 1 milliliter phosphate buffered saline pH 7.2 three times. Finally, glutathione functionalized crosslinked iron oxide particles linked to bovine serum albumin were stored in 0.2 milliliter phosphate buffered saline pH 7.2 at 4° C.

The final product was brown in colour and paramagnetic in nature. The presence of bovine serum albumin was evaluated using recovery of drugs (see example 54). Drug recovery was not observed in the absence of bovine serum albumin.

### Example 37

Linking of Glutathione Functionalized Crosslinked Iron Oxide Particles to N-Acetyl Glucosamine

10 milligrams crosslinked iron oxide particles of example 14 and 1 milligram N-acetyl glucosamine were dispersed in 1 milliliter distilled water. 1 milligram N-(3-dimethylaminopropyl)-N′-ethylcarbodiimide hydrochloride and 100 microgram 4-dimethylamino pyridine in 1 milliliter distilled water was added to the above solution. The reaction mixture was vortexed for 3 hours. The solids were separated using a magnet and washed with 1 milliliter distilled water three times. Finally, glutathione functionalized iron oxide particles linked to N-acetyl glucosamine were dried in oven at 60° C. and stored in dried state at 4° C.

The final product was brown in colour and paramagnetic in nature. The spectral peaks in Fourier transform infrared spectroscopy at 1467 cm⁻¹ confirmed the presence of ester group, thereby confirming the linking of N-acetyl glucosamine to glutathione functionalized crosslinked iron oxide particles. The presence of N-acetyl glucosamine was further confirmed by protein recovery assay (see example 55). Protein recovery was not observed using glutathione functionalized crosslinked iron oxide particles without N-acetyl glucosamine.

### Example 38

Linking of (3-aminopropyl)silane Functionalized Glass Beads Crosslinked with Glutaraldehyde to Anti-Epithelial Cell Adhesion Molecule Antibody

100 milligrams glass beads of example 16 were treated with 1 microgram of anti-epithelial cell adhesion molecule antibody in 0.2 milliliter phosphate buffered saline pH 7.2 and vortexed at 4° C. for 4 hours. The reaction product was washed with 1 milliliter phosphate buffered saline pH 7.2 three times and final product was stored in 0.2 milliliter phosphate buffered saline pH 7.2 at 4° C. The final product was transparent in nature.

### Example 39

Linking of (3-aminopropyl)silane Functionalized Glass Beads Crosslinked with Glutaraldehyde to Transferrin

100 milligrams glass beads of example 16 were treated with 1 microgram transferrin in 0.2 milliliter phosphate buffered saline pH 7.2, followed by vortexing at 4° C. for 4 hours. Thereafter, the reaction product was washed with 1 milliliter phosphate buffered saline pH 7.2 three times and final product was stored in 0.2 milliliter phosphate buffered saline pH 7.2 at 4° C. The final product was transparent in nature.

### Example 40

Linking of (3-aminopropyl)silane Functionalized Glass Beads Crosslinked with Glutaraldehyde to Bovine Serum Albumin

100 milligrams of glass beads of example 16 were treated with 1 microgram bovine serum albumin in 0.2 milliliter phosphate buffered saline pH 7.2, and vortexed at 4° C. for 4 hours. The reaction product was washed with 1 milliliter phosphate buffered saline pH 7.2 three times and the product was stored in 0.2 milliliter phosphate buffered saline pH 7.2 at 4° C. The final product was transparent in nature.

### Example 41

Linking of (3-aminopropyl)silane Functionalized Glass Beads Crosslinked with Glutaraldehyde to N-acetyl Glucosamine

To 100 milligrams glass beads of example 16 in 2 milliliter methanol, was added a drop of acetic acid and the reaction mixture was vortexed at room temperature for 15 minutes. Methanol was decanted and solids were washed with 1 milliliter distilled water three times. These glass beads were poured in 0.2 milliliter phosphate buffered saline pH 7.2 containing 100 micrograms N-acetyl glucosamine and vortexed at 4° C. for 4 hours. The reaction product was washed with 1 milliliter phosphate buffered saline pH 7.2 three times and was stored in 0.2 milliliter phosphate buffered saline pH 7.2 at 4° C. The final product was transparent in nature.

### Example 42

Crosslinking of Iron Oxide Nanoparticles with (3-mercaptopropyl)silane Functionalized Glass Beads Followed by Linking with Glutathione

100 milligram glass beads functionalized with (3-mercaptopropyl)silane were sonicated with 10 milligram iron oxide nanoparticles of example 1 in 3 milliliter of distilled water and 1 milliliter of methanol for 1 hour. Iron oxide nanoparticles crosslinked with glass beads were washed with 1 milliliter distilled water three times and supernatant was removed. 1 milligram glutathione was added in 3 milliliter of distilled water and 1 milliliter of methanol and reaction mixture was stirred for 1 hour. The reaction was washed with 1 milliliter distilled water thrice and the reaction product was dried in oven at 60° C. and stored in distilled water at 4° C.

The crosslinking of (3-mercaptopropyl)silane functionalized glass beads with iron oxide nanoparticles followed by linkage to glutathione was confirmed by change in glass bead color to brown and paramagnetic nature of the product.

### Example 43

Linking of Glutathione Functionalized Crosslinked Iron Oxide Particles to poly(amidoamine) Dendrimer

50 milligram crosslinked iron oxide particles of example 14 were dispersed in 20 milliliter 2-(N-morpholino)ethanesulfonic acid buffer pH 6.0. A solution of 7.7 milligram N-(3-dimethylaminopropyl)-N′-ethylcarbodiimide hydrochloride and 7.0 milligram N-hydroxysuccinamide was added to the above dispersion and stirred for 1 hour. The dispersion was separated using a magnet and washed with phosphate buffered saline pH 7.4. 5 milligram poly(amidoamine) G4 (Aldrich Sigma Catalogue no 412449) was added in 1 milliliter of phosphate buffered saline pH 7.4 and reaction mixture was vortexed for 4 hours. The reaction product was separated by placing the reaction flask on magnet and decanting the supernatant. 10 milliliter ethanol was added to the flask and particles were dispersed. The washing was repeated with 10 milliliter ethanol and 10 milliliter of distilled water alternatively until the supernatant was colourless. The glutathione functionalized crosslinked iron oxide particles linked to poly-(amidoamine) were dried in oven at 60° C. and stored at 4° C.

These particles were brown in colour and were paramagnetic. The Fourier transform infrared spectroscopy which showed a spectral peak at 3386 cm⁻¹ confirmed the presence of amine group (—NH₂), thereby confirmed the linking of glutathione functionalized crosslinked iron oxide particles with poly-(amidoamine) (PAMAM). The recovery of deoxyribose nucleic acid was estimated using poly-(amidoamine) (PAMAM) linked glutathione functionalized crosslinked iron oxide particles (example 56). The crosslinked iron oxide particles without poly(amidoamine) did not capture deoxyribose nucleic acid.

### Example 44

Crosslinking of (3-aminopropyl)silane Functionalized Glass Beads with Iminothiolane Functionalized Iron Oxide Nanoparticles Followed by Linking with Iminothiolane Spacer and Linking to Anti-Epithelial Cell Adhesion Molecule Antibody

100 milligram glass beads of example 19 were treated with 1 microgram of anti-epithelial cell adhesion molecule antibody in the presence of 40 microgram iminothiolane hydrochloride dissolved in 0.2 milliliter phosphate buffered saline pH 7.2 and incubated for 4 hours at 4° C. The product was washed with 1 milliliter phosphate buffered saline pH 7.2 three times and finally stored in 0.2 milliliter phosphate buffered saline pH 7.2 at 4° C.

The final product was brown in colour and paramagnetic.

### Example 45

Crosslinking of (3-aminopropyl)silane Functionalized Glass Beads with Glutathione Functionalized Iron Oxide Nanoparticles and Linking to Anti-Epithelial Cell Adhesion Molecule Antibody to the Later

100 milligrams glass beads crosslinked with glutathione functionalized iron oxide nanoparticles of example 21 were treated with 383 micrograms N-(3-dimethylaminopropyl)-N′-ethylcarbodiimide hydrochloride and 230 micrograms N-hydroxysuccinimide dissolved in 0.2 milliliter 2-(N-morpholino)ethanesulfonic acid pH 6.0 and incubated at room temperature for 1 hour. The reaction product was filtered and washed with 1 milliliter distilled water thrice and poured in 0.2 milliliter phosphate buffered saline pH 7.2 containing 1 microgram of anti-epithelial cell adhesion molecule antibody, followed by vortexing for 4 hours at 4° C. The product was washed with 1 milliliter phosphate buffered saline pH 7.2 thrice and stored in 0.2 milliliter phosphate buffered saline pH 7.2 at 4° C. The final product was brown in colour and paramagnetic.

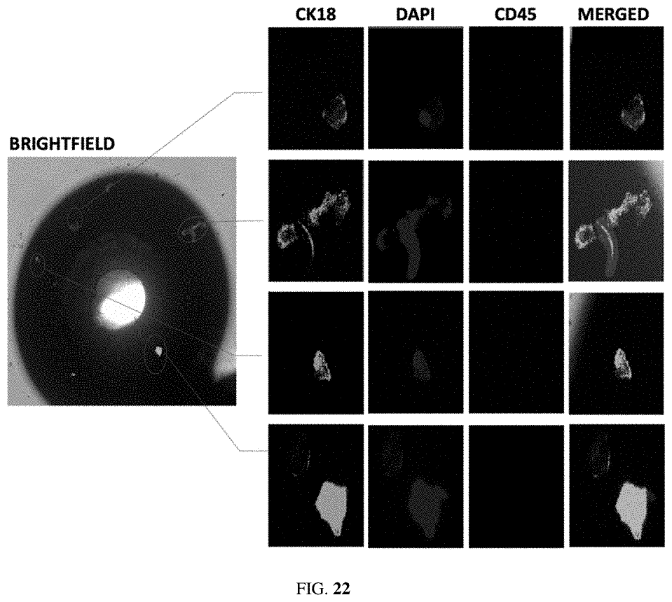

The linkage of anti-epithelial cell adhesion molecule antibody was confirmed by CTC capturing assay (see example 59). FIG. 22 shows the capture of CTCs. CTCs were not captured when the anti-epithelial cell adhesion molecule antibody was absent on these beads.

### Example 46

Crosslinking of (3-aminopropyl)silane Functionalized Glass Beads with Glutathione Functionalized Iron Oxide Nanoparticles and Linking to Transferrin

100 milligrams (3-aminopropyl)silane functionalized glass beads crosslinked with glutathione functionalized iron oxide nanoparticles of example 21 was treated with 383 micrograms N-(3-dimethylaminopropyl)-N′-ethylcarbodiimide hydrochloride and 230 micrograms N-hydroxysuccinimide dissolved in 0.2 milliliter phosphate buffered saline pH 6.0 and incubated for 1 hour. The reaction mixture was filtered and the residue was washed with 0.2 milliliter distilled water three times and poured in 0.2 milliliter phosphate buffered saline pH 7.2 containing 1 microgram transferrin and vortexed for 4 hours at 4° C. The product was finally washed with 1 milliliter phosphate buffered saline pH 7.2 three times and stored in 0.2 milliliter phosphate buffered saline pH 7.2 at 4° C.

The final product was brown in colour and paramagnetic.

### Example 47

(3-aminopropyl)silane Functionalized Glass Beads Crosslinked with Glutathione Functionalized Iron Oxide Nanoparticles and Linking to Bovine Serum Albumin

100 milligrams glass beads crosslinked with iron oxide nanoparticles of example 21 were treated with 383 micrograms N-(3-dimethylaminopropyl)-N′-ethylcarbodiimide hydrochloride and 230 micrograms N-hydroxysuccinimide dissolved in 0.2 milliliter phosphate buffered saline pH 6.0 and incubated at room temperature for 1 hour. The reaction mixture was filtered and washed with 1 milliliter distilled water three times and the product was poured in 0.2 milliliter phosphate buffered saline pH 7.2 containing 1 microgram bovine serum albumin followed by vortexing for 4 hours at 4° C. The reaction product was finally washed with 1 milliliter phosphate buffered saline pH 7.2 three times and stored in 0.2 milliliter phosphate buffered saline pH 7.2 at 4° C. The final product was brown and paramagnetic.

The presence of bovine serum albumin was confirmed using drug recovery assay (see example 54). Drug recovery was not observed when bovine serum albumin was not linked to the glass beads of this example.

### Example 48

Crosslinking of (3-aminopropyl)silane Functionalized Glass Beads with Glutathione Functionalized Iron Oxide Nanoparticles and Linking to N-acetyl Glucosamine

100 milligrams glass beads crosslinked with iron oxide nanoparticles of example 21 and 1 milligram N-acetyl glucosamine were added to a solution of 2 milligram N-(3-dimethyl aminopropyl)-N′-ethylcarbodiimide hydrochloride and 100 microgram dimethylaminopyridine in 1 milliliter distilled water. The reaction mixture was vortexed for 3 hours. The supernatant was removed and the solid residue was washed with 1 milliliter phosphate buffered saline pH 7.2 three times. The reaction product was dried in oven at 60° C. and stored at 4° C. The final product was brown in colour and paramagnetic.

The presence of N-acetyl glucosamine on the beads was confirmed be protein recovery assay (see example 55). Protein recovery was not observed when N acetyl glucosamine was not present.

### Example 49

Crosslinking of (3-aminopropyl)silane Functionalized Glass Beads with Citric Acid Functionalized Iron Oxide Nanoparticles Linked with 12-Aminododecanoic Acid Followed by Linking with Anti-Epithelial Cell Adhesion Molecule Antibody

100 milligram glass beads crosslinked with iron oxide nanoparticles of example 23 were incubated for 1 hour with 383 microgram N-(3-dimethylaminopropyl)-N′-ethylcarbodiimide hydrochloride and 230 microgram N-hydroxysuccinimide dissolved in 0.2 milliliter 2-(N-morpholino) ethanesulfonic acid pH 6.0. The reaction mixture was filtered and the residue was washed with 1 milliliter distilled water thrice and poured in 0.2 milliliter phosphate buffered saline pH 7.2 containing 1 microgram of anti-epithelial cell adhesion molecule antibody and vortexed for 4 hours at 4° C. The reaction product was washed with 1 milliliter phosphate buffered saline pH 7.2 three times and stored in 0.2 milliliter phosphate buffered saline pH 7.2 at 4° C. The final product was brown in colour and paramagnetic.

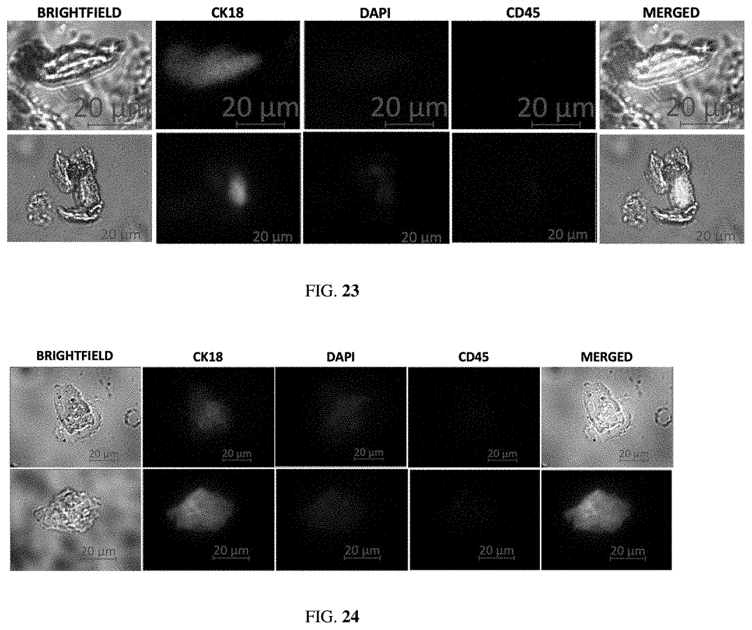

The linkage of anti-epithelial cell adhesion molecule antibody was confirmed by CTC capture assay (see example 59). FIG. 23 shows the capture of CTCs on glass beads to which anti-epithelial cell adhesion molecule antibody was linked. CTCs were not captured when (3-aminopropyl)silane functionalized glass beads cross linked with citric acid functionalized iron oxide nanoparticles were not linked with anti-epithelial cell adhesion molecule antibody.

### Example 50

Crosslinking of (3-glycidyloxypropyl)silane Functionalized Glass Beads Linked to poly(amidoamine) Dendrimer with Glutathione Functionalized Iron Oxide Nanoparticles Followed by Linking with Anti-Epithelial Cell Adhesion Molecule Antibody

200 milligram glass beads of example 11 were added to the 2 milligrams poly(amidoamine) dendrimer in 200 microliter dimethylformamide and reaction mixture was vortexed for 3 hours. The reaction mixture was washed with 1 milliliter distilled water three times. 1 milligram glutathione functionalized iron oxide nanoparticles of example 2 were dispersed in 1 milliliter sodium carbonate buffer pH 9.3. The reaction mixture was sonicated for 5 minutes and then vortexed for 3 hours. The product was washed with 1 milliliter distilled water thrice. To this mixture, 383 micrograms N-(3-dimethylaminopropyl)-N′-ethylcarbodiimide hydrochloride and 230 micrograms N-hydroxysuccinimide dissolved in 0.2 milliliter phosphate buffered saline pH 6.0 and the content was vortexed for 1 hour. The reaction mixture was washed with 3 milliliter of phosphate buffered saline. 1 microgram anti-epithelial cell adhesion molecule antibody in 0.2 milliliter phosphate buffered saline pH 7.4 was added to the above and vortexed for 4 hours at 4° C. After 4 hours, the reaction mixture was washed with phosphate buffered saline pH 7.4 thrice and product was finally stored in 0.2 milliliter phosphate buffered saline pH 7.4 at 4° C.

The Fourier transform infrared spectrum with spectral peak at 3406 and 1625 cm⁻¹ confirmed the presence of free —NH₂ and amide functionality from poly(amidoamine) (PAMAM) dendrimer linked to silanized glass bead.

### Example 51

Crosslinking of (3-glycidyloxypropyl)silane Functionalized Glass Beads Linked to Bovine Serum Albumin with Glutathione Functionalized Iron Oxide Nanoparticles and Linking to Anti-Epithelial Cell Adhesion Molecule Antibody

200 milligram glass beads of example 11 were incubated with 1 microgram of bovine serum albumin in 1 milliliter sodium carbonate buffer pH 9.2 for 4 hours at 4° C. The reaction mixture was washed with 1 milliliter sodium carbonate buffer pH 9.2 three times and 1 milligram iron oxide nanoparticle of example 2 was added in 1 milliliter sodium carbonate buffer pH 9.2 and incubated for 4 hours. The supernatant was separated and the residue was washed with 1 milliliter phosphate buffered saline pH 7.2 thrice. Further, 1 microgram anti-epithelial cell adhesion molecule antibody was added to the reaction mixture. To this mixture, 383 micrograms N-(3-dimethylaminopropyl)-N′-ethylcarbodiimide hydrochloride and 230 micrograms N-hydroxy succinimide dissolved in 0.2 milliliter phosphate buffered saline pH 6.0 was added and vortexed for 4 hours at 4° C. The reaction mixture was washed with 3 milliliter of phosphate buffered saline pH 7.2. The reaction product was finally stored in 0.2 milliliter phosphate buffered saline pH 7.4 at 4° C. The final product was brown and paramagnetic. The linkage of anti-epithelial cell adhesion molecule antibody was confirmed by capturing CTCs (see example 59). FIG. 24 shows the capture of CTCs.

### Example 52

Linking of Glutathione Functionalized Iron Oxide Nanoparticles with Transferrin and Anti-Epithelial Cell Adhesion Molecule Antibody

10 milligram glutathione functionalized iron oxide particles of example 2 were dispersed in 1 milliliter 2-(N-morpholino) ethanesulfonic acid buffer pH 6.0. 383 microgram N-(3-dimethylaminopropyl)-N′-ethylcarbodiimide hydrochloride and 230 microgram N-hydroxysuccinimide in 1 milliliter 2-(N-morpholino)ethanesulfonic acid buffer pH 6.0 was added to the above mixture followed by vortexing for 1 hour. Thereafter the particles were isolated using magnet and washed with 1 milliliter phosphate buffered saline pH 7.2. The particles were re-dispersed in 1 milliliter phosphate buffered saline pH 7.2 and 1 microgram transferrin added to the reaction mixture, and vortexed for 4 hours at 4° C. The particles were isolated using magnet and washed with 1 milliliter phosphate buffered saline pH 7.2 three times. The product was resuspended in 1 milliliter 2-(N-morpholino)ethanesulfonic acid buffer pH 6.0. 383 micrograms N-(3-dimethylaminopropyl)-N′-ethylcarbodiimide hydrochloride and 230 micrograms N-hydroxysuccinimide in 1 milliliter 2-(N-morpholino) ethanesulfonic acid buffer 6.0 was added to the above mixture followed by vortexing for 1 hour. Thereafter the particles were isolated using magnet and washed with 1 milliliter phosphate buffered saline pH 7.2. The particles were re-dispersed in 1 milliliter phosphate buffered saline pH 7.2 and 1 microgram anti-epithelial cell adhesion molecule antibody was added to the reaction mixture, and vortexed for 4 hours at 4° C. The particles were isolated using magnet and washed with 1 milliliter phosphate buffered saline pH 7.2 three times. The final product was stored in 0.2 milliliter phosphate buffered saline pH 7.2 at 4° C.

The iron oxide nanoparticles linked to transferrin and anti-epithelial cell adhesion molecule antibody were brown in colour and paramagnetic in nature. The linkage of anti-epithelial cell adhesion molecule antibody and transferrin was confirmed by capturing CTCs (see example 59). FIG. 25 shows the capture of CTCs using transferrin and anti-epithelial cell adhesion molecule antibody linked iron oxide nanoparticles.

### Example 53

Linking of (3-glycidyloxypropyl)silane Functionalized Glass Beads with Transferrin and Anti-Epithelial Cell Adhesion Molecule Antibody

200 milligram glass beads of example 11 were treated with 1 microgram transferrin in 1 milliliter sodium carbonate buffer pH 9.2 for 4 hours at 4° C. The glass beads were washed with 1 milliliter sodium carbonate buffer pH 9.2 three times and were treated with 1 microgram anti-epithelial cell adhesion molecule in 1 milliliter sodium carbonate buffer pH 9.2 for 4 hours at 4° C. The glass beads were washed with 1 milliliter phosphate buffered saline pH 7.2 three times and finally stored in 0.2 milliliter phosphate buffered saline pH 7.2 at 4° C.

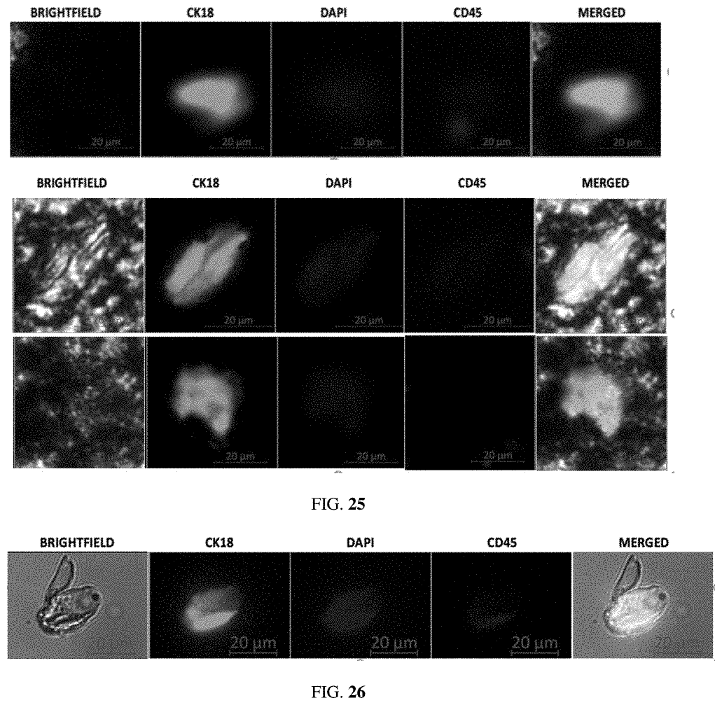

The (3-glycidyloxypropyl)silane functionalized glass beads linked to transferrin and anti-epithelial cell adhesion molecule antibody were transparent. The linkage of anti-epithelial cell adhesion molecule antibody and transferrin was confirmed by capturing CTCs (see example 59). FIG. 26 shows the capture of CTCs on transferrin and anti-epithelial cell adhesion molecule antibody linked (3-glycidyloxypropyl)silane functionalized glass beads. CTCs were not captured when the anti-epithelial cell adhesion molecule antibody and transferrin were absent on (3-glycidyloxypropyl)silane functionalized glass beads.

### Example 54

Recovery of Drugs Using Iron Oxide

100 microgram/milliliter of vancomycin hydrochloride and 50 microgram/milliliter metformin hydrochloride were individually dissolved in 1 milliliter phosphate buffered saline of pH 7.4. 10 milligram iron oxide nanoparticles of examples 26 and 36 were individually incubated up to 12 hours at room temperature. The iron oxide nanoparticles were separated by magnet and free vancomycin hydrochloride/metformin hydrochloride in the supernatant was analyzed by UV-Vis spectra at λₘₐₓ=281/234 nanometer. The iron oxide nanoparticles of example 26 and 36 recovered 4 and 3 micrograms of vancomycin hydrochloride per milligram, respectively. The iron oxide nanoparticles of example 26 and 36 recovered 8 and 6.8 microgram of metformin hydrochloride, per milligram of the composition, respectively. Similar experiment were carried out for recovering drugs from blood.

Iron oxide nanoparticles comprising serum albumin were incubated with 1 milliliter of blood for 10 minutes. The iron oxide nanoparticles were separated by magnet and 1 milliliter of red blood cell lysis buffer was added and mixed and stirred with sodium chloride buffer for 5 minutes. The drug in the supernatant was analyzed using UV-Vis spectroscopy.

Recovery of Drugs Using Glass Beads

100 microgram/milliliter of vancomycin hydrochloride and 50 microgram/milliliter metformin hydrochloride were dissolved in 1 milliliter phosphate buffered saline of pH 7.4 respectively. 100 milligram glass beads of examples 31 and 47 were incubated u to 12 hours at room temperature. The glass beads were decanted and free vancomycin hydrochloride/metformin hydrochloride in the supernatant was analyzed by UV-Vis spectra at λₘₐₓ=281/234 nanometer. The glass beads of example 31 and 47 recovered 0.30 and 0.35 micrograms of vancomycin hydrochloride per milligram of the sample respectively. The glass beads of example 31 and 47 recovered 0.22 and 0.30 microgram of metformin hydrochloride per milligram of the sample respectively. Similar experiment were carried out for recovering drugs from blood.

Glass beads comprising bovine serum albumin were incubated with 1 milliliter of blood for 10 minutes. The glass beads were separated by decantation and 1 milliliter of red blood cell lysis buffer was added and mixed and stirred with sodium chloride buffer for 5 minutes. The drug in the supernatant was analyzed using UV-Vis spectroscopy.

### Example 55

Recovery of Bovine Serum Albumin Using Iron Oxide Nanoparticles

Bovine serum albumin (500 microgram/milliliter) was solubilized in phosphate buffered saline pH 7.4 and incubated with 10 milligram iron oxide samples of example 27 and 37 for 12 hours at room temperature. The iron oxide nanoparticles were separated by magnet and free bovine serum albumin in the supernatant was analyzed by UV-Vis spectra at λₘₐₓ=280 nanometer. The iron oxide nanoparticles of example 27 and 37 recovered 8.2 and 7.2 microgram of bovine serum albumin per milligram of the sample respectively. Similar experiments were carried out for recovering bovine serum proteins from blood.

Iron oxide nanoparticles comprising N-acetylglucosamine were incubated with 01 milliliter of blood for 10 minutes. The iron oxide nanoparticles were separated by magnet and 1 milliliter of red blood cell lysis buffer was added and mixed and stirred with sodium chloride buffer for 05 minutes. The protein in the supernatant was analyzed using UV-Vis spectroscopy.

Recovery of Bovine Serum Albumin Using Glass Beads

Bovine serum albumin (500 microgram/milliliter) was solubilized in phosphate buffered saline pH 7.4 and incubated with 100 milligram glass beads of example 32 and 48 for 12 hours at room temperature. The glass beads were decanted and free bovine serum albumin in the supernatant was analyzed by UV-Vis spectra at λₘₐₓ=280 nanometer. The glass beads of example 32 and 48 recovered 0.7 and 0.62 microgram of bovine serum albumin per milligram of the sample respectively. Similar experiments were carried out for recovering serum proteins from blood.

Glass beads comprising N-acetylglucosamine were incubated with 1 milliliter of blood for 10 minutes. The glass beads were separated by decantation and 1 milliliter of red blood cell lysis buffer was added and mixed and stirred with sodium chloride buffer for 5 minutes. The protein in the supernatant was analyzed using UV-Vis spectroscopy.

### Example 56

Recovery of Deoxyribonucleic Acid Using Iron Oxide Nanoparticles

The iron oxide nanoparticles linked to poly(amidoamine) dendrimer of example 9, 43 were used for the recovery of deoxyribonucleic acid. 15.62 micrograms deoxyribonucleic acid was solubilized in 1 milliliter phosphate buffered saline pH 7.4 and incubated with 1 milligram iron oxide samples of examples 9 and 43 respectively for 12 hours at room temperature. The iron oxide nanoparticles were separated by magnet and free deoxyribonucleic acid in the supernatant was analyzed by UV-Vis spectra at λₘₐₓ=260 nanometer. The iron oxide samples of example 9 and 43 recovered 13.2 and 12.8 microgram of deoxyribonucleic acid per milligram of the sample respectively. Similar experiments were carried out for recovering deoxyribonucleic acid from blood.

Iron oxide nanoparticles comprising poly (amidoamine) dendrimer were incubated with 1 milliliter of blood for 10 minutes. The iron oxide nanoparticles were separated by magnet and 1 milliliter of red blood cell lysis buffer was added and mixed and stirred with sodium chloride buffer for 5 minutes. The deoxyribonucleic acid in the supernatant was analyzed using UV-Vis spectroscopy.

Glass Beads

Recovery of Deoxyribonucleic Acid Using Glass Beads

15.62 micrograms deoxyribonucleic acid was solubilized in 1 milliliter phosphate buffered saline pH 7.4 and incubated with 100 milligram glass beads of example 10 for 12 hours at room temperature. The glass beads were decanted and free deoxyribonucleic acid in the supernatant was analyzed by UV-Vis spectra at λₘₐₓ=260 nanometer. The glass beads of example 10 recovered 0.1 microgram of deoxyribonucleic acid per milligram of the sample. Similar experiments were carried out for recovering deoxyribonucleic acid from blood.

Glass beads comprising poly (amidoamine) dendrimer were incubated with 1 milliliter of blood for 10 minutes. The glass beads were separated by decantation and 1 milliliter of red blood cell lysis buffer was added and mixed and stirred with sodium chloride buffer for 5 minutes. The deoxyribonucleic acid in the supernatant was analyzed using UV-Vis spectroscopy.

### Example 57

Red Blood Cells Hemolysis Assay

Red Blood Cells from whole human blood (5 milliliter) were separated by centrifugation (500×g, 5 minutes) at room temperature and washed in 150 mM sodium chloride (NaCl) solution two times and resuspended in a total final volume of 5 milliliter with phosphate buffered saline with pH 7.4. The resuspended red blood cells were incubated by mixing with materials listed in Table 1 for 30 minutes at 37° C. The samples were centrifuged (500×g, 5 minutes, room temperature), and supernatants were collected and analyzed for hemolysis using UV spectroscopy at spectra 540 nm. The percentage hemolysis was estimated against a negative control phosphate buffered saline with pH 7.4 and positive control 0.5% Triton-X100. The percentage hemolysis is shown in Table 1.

TABLE 1
Percentage hemolysis with Red Blood Cells for
examples, raw materials, and intermediates.

| Sr. | Sample Name and amount in percentage/ | Percentage |
|---|---|---|
| No. | milligrams (mg) | Hemolysis |
| 1. | Phosphate Buffered Saline | 0% |
| 2. | Triton-X100 (0.5%) | 100% |
| 3. | Glutathione (1 mg) | 0% |
| 4. | Glutathione (10 mg) | 0% |
| 5. | Citric Acid (1 mg) | 0% |
| 6. | Citric Acid (4 mg) | 30% |
| 7. | 0.5% (3-Aminopropyl)triethoxysilane | 0% |
| 8. | 10% (3-Aminopropyl)triethoxysilane | 36% |
| 9. | 0.5% (3-Glycidyloxypropyl)trimethoxysilane | 0% |
| 10. | 10% (3-Glycidyloxypropyl)trimethoxysilane | 63% |
| 11. | Poly(amidoamine) (1 mg) | 0% |
| 12. | Poly(amidoamine) (10 mg) | 0.9% |
| 13. | 12-Aminododecanoic Acid (1 mg) | 0% |
| 14. | 12-Aminododecanoic Acid (10 mg) | 0% |
| 15. | N-acetyl glucosamine (1 mg) | 0% |
| 16. | N-acetyl glucosamine (4 mg) | 0% |
| 17. | Succinic Anhydride (4 mg) | 100% |
| 18. | Unwashed Glass Beads (2 mm) (~200 mg) | 0.9% |
| 19. | Washed Glass Beads (2 mm) (~200 mg) | 0% |
| 20 | Example 1 (1 mg) | 0% |
| 21. | Example 1 (10 mg) | 0.8% |
| 22. | Example 2 (1 mg) | 0% |
| 23. | Example 5 (10 mg) | 0% |
| 24. | Example 6 (10 mg) | 0% |
| 25. | Example 7 (10 mg) | 0% |
| 26. | Example 10 (~200 mg) | 0% |
| 27. | Example 11 (~200 mg) | 0% |
| 28. | Example 13 (~60 mg) | 0% |
| 29. | Example 14 (10 mg) | 0% |
| 30. | Example 19 (~60 mg) | 0% |
| 31. | Example 21 (~60 mg) | 0% |
| 32. | Example 22 (~60 mg) | 0% |
| 33. | Example 23 (~60 mg) | 0% |
| 34. | Example 27 (10 mg) | 0% |
| 35. | Example 32 (~60 mg) | 0% |
| 36. | Example 51 (~60 mg) | 0% |

### Example 58

Human Colon Cancer Cells (HCT116) Capture

HCT116 cells were incubated for 5 minutes with samples prepared as per example 28. Anti-epithelial cell adhesion molecule antibody was linked to 1) glass coverslip (2 milligrams), 2) hollow glass capillary tubes (2 milligrams) and 3) glass beads (60 milligrams). The enriched CTCs were fixed with absolute ethanol and immunostained with antibodies against cytokeratin (CK-18) and leucocyte common antigen (CD-45) and with nuclear-staining probe 4′,6-diamidino-2-phenylindole (DAPI). Isolated cells were imaged and observed under fluorescence microscopy (FIG. 14, 15). The HCT 116 cell capture is summarized in Table 2.

TABLE 2

| | | | Cell Type |
|---|---|---|---|
| No. | Example | Description | Captured |
| 1. | 24 | Glutathione functionalized iron oxide nanoparticles linked with | CTC |
| | | anti-epithelial cell adhesion molecule antibody | |
| 2. | 25 | Glutathione functionalized iron oxide nanoparticles linked with | CTC |
| | | transferrin | |
| 3. | 28 | (3-glycidyloxypropyl)silane functionalized glass cover slip linked | HCT- |
| | | with anti-epithelial cell adhesion molecule antibody | 116 |
| 4. | 28 | (3-glycidyloxypropyl)silane functionalized glass beads linked with | HCT- |
| | | anti-epithelial cell adhesion molecule antibody | 116 |
| 5. | 28 | (3-glycidyloxypropyl)silane functionalized glass cover slip linked | CTC |
| | | with anti-epithelial cell adhesion molecule antibody | Cluster |
| 6. | 30 | (3-aminopropyl)silane functionalized glass beads linked with | CTC |
| | | glutathione to transferrin | |
| 7. | 33 | Glutathione functionalized crosslinked iron oxide nanoparticles | CTC |
| | | linked with anti-epithelial cell adhesion antibody | |
| 8. | 35 | Glutathione functionalized crosslinked iron oxide nanoparticles | CTC |
| | | linked with transferrin | |
| 9. | 45 | (3-aminopropyl)silane functionalized glass beads linked with | CTC |
| | | glutathione functionalized iron oxide nanoparticles and linking | |
| | | anti-epithelial cell adhesion molecule antibody to the later | |
| 10. | 49 | Crosslinked (3-aminopropyl)silane functionalized glass beads | CTC |
| | | with citric acid functionalized iron oxide nanoparticles linked with | |
| | | 12-aminododecanoic acid and linking to anti-epithelial cell | |
| | | adhesion molecule antibody | |
| 11. | 51 | Glutathione functionalized iron oxide nanoparticles linked with | CTC |
| | | bovine serum albumin and anti-epithelial cell adhesion molecule | |
| | | antibody | |
| 12. | 52 | Glutathione functionalized crosslinked iron oxide nanoparticles | CTC |
| | | linked with transferrin and anti-epithelial cell adhesion molecule | |
| | | antibody | |
| 13. | 53 | (3-Glycidyloxypropyl)silane functionalized glass beads linked | CTC |
| | | with transferrin and anti-epithelial cell adhesion molecule | |
| | | antibody | |

Capturing CTCs and HCT 116 Cells.

The cell source for compositions of serial numbers 3 and 4 was cell/tissue culture. For all others it was human blood.

### Example 59

Capture of Circulating Tumor Cells (CTC) from Cancer Patient's Blood

Cancer patient blood sample 1.5 milliliter was incubated for 5 minutes with materials of examples listed in Table 2. In the case of magnetic nanoparticles, CTCs were enriched using magnetic separation. In the case of glass beads, the supernatants were decanted off. Captured circulating tumor cells were fixed with absolute ethanol and immuno-stained with cytokeratin (CK-18), leucocyte common antigen (CD-45) and with nuclear-staining probe 4′,6-diamidino-2-phenylindole (DAPI). Captured cells were observed and imaged under fluorescence microscopy (see FIG. 16-26).

### Example 60

Circulating Tumor Cells Capture in a Series of Trap Devices Using Cancer Patient Whole Blood

Cancer patient blood sample 3 milliliter was incubated in first device trap with samples of example 24 (300 micrograms) for 5 minutes. The sample was separated using magnet and the blood was then re-transferred into a second trap having fresh 300 microgram of material from example 24 for 5 minutes. The material with cancer was separated using magnet and the blood was then re-transferred into third trap having fresh 300 microgram of material from Example 24 for 05 minutes. The material with cancer cells was separated using magnet and CTCs were enriched, fixed with absolute ethanol and immuno-stained with cytokeratin (CK-18), leucocyte common antigen (CD-45) and with nuclear-staining probe 4′,6-diamidino-2-phenylindole (DAPI). Captured cells were observed and imaged under fluorescence microscopy. The efficiency of capturing circulating cells was calculated in each trap and the total number of cells were accounted (FIG. 12). The CTC cell capture in each trap device using cancer patient whole blood was 47 (70%), 13 (19%), and 7(11%) for CTC traps 1, 2, and 3, respectively.

### Example 61

Circulating Tumor Cells Capture in Series of Trap Device Using Cancer Patient Whole Blood

Cancer patient's blood sample of volume 3 milliliter was distributed into three equal volumes and incubated in three separate trap devices each with example 24 (300 micrograms) linked with anti-epithelial cell adhesion molecule antibody and incubated for 5 minutes. The materials with cancer cells was separated using magnet and CTCs were enriched, fixed with absolute ethanol and immuno-stained with cytokeratin (CK-18), leucocyte common antigen (CD-45) and with nuclear-staining probe 4′,6-diamidino-2-phenylindole (DAPI). Captured cells were observed and imaged under fluorescence microscopy. The efficiency of capturing circulating cells using 3 milliliter of blood distributed in each trap and the total number of CTCs were accounted to be 136 (FIG. 13). The CTCs cell capture in each trap device using cancer patient whole blood was 51, 43, and 42 in traps 1, 2, and 3, respectively.

### Example 62

Destruction of Circulating Tumor Cells Using Cisplatin

1.5 ml blood of a cancer patient was incubated with 300 micrograms of composition of example 33 for 5 minutes. CTCs were isolated using magnetic separation and cells were fixed with absolute ethanol and immuno-stained with cytokeratin (CK-18), leucocyte common antigen (CD-45) and with nuclear-staining probe 4′,6-diamidino-2-phenylindole (DAPI). Captured cells were imaged under fluorescence microscope, wherein 3 CTCs were isolated.

1.5 ml blood of a cancer patient was incubated with 300 micrograms of composition of example 33 for 5 minutes. CTCs were isolated using magnetic separation and incubated for two hours with 30 micrograms Cisplatin in 100 microliter phosphate buffered saline, pH 7.4, and the composition was fixed with absolute ethanol and immuno-stained with cytokeratin (CK-18), leucocyte common antigen (CD-45) and with nuclear-staining probe 4′,6-diamidino-2-phenylindole (DAPI) and imaged under fluorescence microscope. No CTCs were observed. This shows that the CTCs isolated by the composition of example 33 were destroyed by Cisplatin.

### Exemplary Embodiment

In certain aspects, the disclosure provides a composition comprising: a substrate; and a ligand, wherein the substrate is glass, iron oxide or a combination thereof, and the ligand is at least one of (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, poly(amidoamine) dendrimer, anti-Epithelial Cell Adhesion Molecule—(EpCAM) antibody, transferrin, bovine serum albumin, N-acetylglucosamine or a combination thereof, and wherein the composition is non-hemolytic.

In any aspect or embodiment described herein, the substrate is iron oxide nanoparticles in the size range of from about 10 nm to about 300 nm.

In any aspect or embodiment described herein, the substrate is glass. In any aspect or embodiment described herein, the glass is in the form selected from glass beads, glass capillaries and glass cover slips.

In any aspect or embodiment described herein, the composition further includes a functionalizing agent attached to the substrate covalently or non-covalently. In any aspect or embodiment described herein, the ligand is the functionalizing agent, and is attached to the substrate non-covalently. In any aspect or embodiment described herein, a first portion of the functionalizing agent is linked to a first ligand covalently, and a second portion of the functionalizing agent is linked to a second ligand covalently, wherein the first ligand and the second ligand are not the same. In any aspect or embodiment described herein, the composition further includes a spacer linked to the functionalizing agent. In any aspect or embodiment described herein, the ligand is the spacer, and is attached to the functionalizing agent covalently.

In any aspect or embodiment described herein, the spacer is at least one of glutathione, citric acid, succinic acid, 12 amino dodecanoic acid, iminothiolane, poly (amido amine) dendrimer, cysteine, glutaraldehyde, aspartic acid, mercaptoacetic acid, mercaptopropanoic acid, iminothiolane hydrochloride and dicarboxylic acids, polyimides, poly(amidoamine) (PAMAM), or a combination thereof.

In any aspect or embodiment described herein, the functionalizing agent is selected from (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), glutathione.

In any aspect or embodiment described herein, the description provides a composition comprising a substrate; a functionalizing agent; a spacer; and a ligand, wherein the substrate is glass, iron oxide or a combination thereof, and the ligand is at least one of (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, poly(amidoamine) dendrimer, anti-Epithelial Cell Adhesion Molecule—(EpCAM) antibody, transferrin, bovine serum albumin, N-acetylglucosamine or a combination thereof, and wherein the composition is non-hemolytic.

In any aspect or embodiment described herein, the functionalizing agent is at least one of (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), glutathione or a combination thereof.

In any aspect or embodiment described herein, the spacer is at least one of glutathione, citric acid, succinic acid, 12 amino dodecanoic acid, iminothiolane, poly amidoamine dendrimer, cysteine, glutaraldehyde, aspartic acid, mercaptoacetic acid, mercaptopropanoic acid, iminothiolane hydrochloride, N-γ-maleimidobutyryl-oxysuccinimide ester (GMBS), N-γ-maleimidobutyryl-oxysulfosuccinimide ester (sulfo-GMBS), sulfo-N-hydroxysuccinimide ester (BS(PEG), (PEGylated bis(sulfosuccinimidyl)suberate) or a combination thereof.

In any aspect or embodiment described herein, the spacer is selected from dicarboxylic acids, aliphatic diamines, ω thio carboxylic acid, ω amino carboxylic acids, polyethylene glycol, poly(methacrylic acid) (PMA), poly oligo(ethylene glycol) methacrylate, diglycidyl ether, poly(N-isopropyl acrylaminde) (PNIPAM), poly ω) (PLA), and Polyvinyl alcohol (PVA), in the number average molecular weight from 50 to 50000 kilo Dalton.

In any aspect or embodiment described herein, the ligand is selected from anti-epithelial cell adhesion Molecule antibody, transferrin, bovine serum albumin, and N-acetylglucosamine.

In any aspect or embodiment described herein, the substrate is crosslinked by a crosslinking agent.

In any aspect or embodiment described herein, the crosslinking agent is selected from (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, and iminothiolane.

In any aspect or embodiment described herein, the substrate is magnetic iron oxide particle crosslinked by a cross linking agent, wherein the crosslinked magnetic iron oxide particle does not cause interference during imaging.

In any aspect or embodiment described herein, the substrate is glass beads crosslinked by a cross linking agent.

In any aspect or embodiment described herein, the substrate comprises iron oxide and glass, which are crosslinked to each other by a crosslinking agent.

In any aspect or embodiment described herein, the iron oxide substrate and glass substrate, respectively, are linked to a different ligand.

In certain aspects, the disclosure provides methods of crosslinking iron oxide nanoparticles comprising the steps of: functionalizing a first sample of iron oxide nanoparticles with a functionalizing agent (F1); functionalizing a second sample of iron oxide nanoparticles with functionalizing agent (F2); crosslinking the first and second samples of iron oxide nanoparticles with a crosslinking agent; isolating the crosslinked iron oxide particles magnetically; and purifying the product.

In any aspect or embodiment described herein, F1 is selected from cysteine, glutathione, 12-aminododecanoic acid, (3-aminopropyl)triethoxysilane (APTES), (3-glycidyloxypropyl) trimethoxysilane (GPTMS), (3-mercaptopropyl)trimethoxysilane (MPTMS), and (3-glycidyloxypropyl)triethoxysilane (GPTES).

In any aspect or embodiment described herein, F2 is selected from cysteine, glutathione, 12-aminododecanoic acid, (3-aminopropyl)triethoxysilane (APTES), (3-glycidyloxypropyl) trimethoxysilane (GPTMS), (3-mercaptopropyl)trimethoxysilane (MPTMS), and (3-glycidyloxypropyl)triethoxysilane (GPTES).

In certain aspects, the disclosure provides methods of crosslinking iron oxide nanoparticles comprising the steps of: dispersing glutathione functionalized iron oxide nanoparticles in distilled water; treating with N-(3-dimethyl aminopropyl)-N′-ethylcarbodiimide hydrochloride and N-hydroxysuccinimide; and recovering glutathione cross-linked iron oxide particles magnetically.

In certain aspects, the disclosure provides methods of crosslinking glass beads comprising the steps of: functionalizing a first sample of glass beads with a functionalizing agent (F1); functionalizing a second sample of glass beads with a functionalizing agent (F2); crosslinking the first and second samples of glass beads with a crosslinking agent; isolating the crosslinked glass beads by gravity separation; and purifying the product.

In any aspect or embodiment described herein, F1 is selected from (3-aminopropyl)triethoxysilane (APTES), (3-glycidyloxypropyl)trimethoxysilane (GPTMS), (3-mercaptopropyl) trimethoxysilane (MPTMS), (3-glycidyloxypropyl)triethoxysilane (GPTES), (3-mercaptopropyl)triethoxysilane (MPTES), glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, and cysteine.

In any aspect or embodiment described herein, F2 is selected from glutathione, mercaptopropanol, mercaptopropionic acid, 12-amino dodecanoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

In certain aspects, the disclosure provides methods of crosslinking glass beads comprising the steps of: functionalizing glass beads with a functionalizing agent (F1); reacting the glass beads with glutaraldehyde (e.g., for about 4 hours) at room temperature followed by the addition of a second sample of glass beads functionalized with a functionalizing agent (F2); reacting (e.g., for about 4 hours) at room temperature, washing with distilled water; and recovering the crosslinked glass beads by drying.

In any aspect or embodiment described herein, F1 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl) triethoxysilane (MPTES).

In any aspect or embodiment described herein, F2 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

In certain aspects, the disclosure provides methods of crosslinking iron oxide nanoparticles with glass beads comprising the steps of: treating iron oxide nanoparticles functionalized with a functionalizing agent (F1) with N-(3-dimethylaminopropyl)-N′-ethylcarbodiimide hydrochloride and N-hydroxysuccinimide in phosphate buffer of pH 6, and reacting the same (e.g., for about 4 hours) at room temperature; adding the functionalized iron oxide nanoparticles to glass beads functionalized with a functionalizing agent (F2) in phosphate buffer of pH 7.2, and reacting the same (e.g., for about 4 hours) at room temperature and treating the reaction mixture with glutaraldehyde (e.g., for about 4 hours); purifying the product by washing with distilled water.

In any aspect or embodiment described herein, F1 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl) triethoxysilane (MPTES).

In any aspect or embodiment described herein, F2 is selected from glutathione, mercaptopropanol, mercaptopropionic acid, 12-aminododeconoic acid, 3-amino-2-(hydroxymethyl) propanoic acid, 4-aminobutanoic acid, 3-amino-3-(4-nitrophenyl) propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

In certain aspects, the disclosure provides methods of recovering drugs from blood comprising the steps of: admixing blood to the non-hemolytic composition as described herein, wherein the ligand is bovine serum albumin; incubating (e.g., for up to about 10 minutes); separating the composition; adding red blood cells lysis buffer; washing the composition and adding sodium chloride buffer; and analysing the supernatant for recovery of a drug by UV-Vis spectroscopy.

In any aspect or embodiment described herein, the drug is selected from Vancomycin, Metformin, Doxorubicin, Methotrexate, Paclitaxel, 5 Fluorouracil, Cisplatin, Camptothecin, Docetaxel, Oxaliplatin, Cyclophosphamide and their pharmaceutically acceptable salts.

In any aspect or embodiment described herein, the non-hemolytic composition substrate is selected from iron oxide nanoparticles, and crosslinked iron oxide nanoparticles, and wherein the non-hemolytic composition is separated by magnetic separation.

In any aspect or embodiment described herein, the non-hemolytic composition substrate is selected from glass beads, cross linked glass beads, iron oxide nanoparticles crosslinked with glass beads, and wherein the non-hemolytic composition is separated by gravity separation.

In certain aspects, the disclosure provides methods of recovering deoxyribonucleic acid from blood comprising the steps of: admixing blood to the non-hemolytic composition as described herein, wherein the ligand comprises poly(amidoamine) dendrimer; incubating (e.g., for up to about 10 minutes); separating the substrate; adding red blood cells lysis buffer; washing the substrate; and analysing the supernatant for deoxyribonucleic acid by UV-Vis spectroscopy.

In any aspect or embodiment described herein, substrate is selected from iron oxide nanoparticles, crosslinked iron oxide nanoparticles, and is separated by magnetic separation.

In any aspect or embodiment described herein, the substrate is selected from glass beads, cross linked glass beads, iron oxide nanoparticles crosslinked with glass beads and is separated by gravity separation.

In certain aspects, the disclosure provides methods of recovering protein from blood comprising the steps of: admixing a composition as described herein comprising N-acetylglucosamine; incubating with blood (e.g., for about 10 minutes); separating the substrate; adding red blood cell lysis buffer; adding sodium chloride buffer; and measuring protein recovered from the supernatant by UV-Vis spectroscopy.

In any aspect or embodiment described herein, the protein is selected from bovine serum albumin, Prealbumin (transthyretin), Alpha 1 antitrypsin, Alpha-1-acid glycoprotein, Alpha-1-fetoprotein, alpha 2-macroglobulin, Gamma globulins, Beta-2 microglobulin, Haptoglobin, Ceruloplasmin, Complement component 3, Complement component 4, C-reactive protein (CRP), Lipoproteins (chylomicrons, VLDL, LDL, HDL), Transferrin, Prothrombin, and Mannose-binding lectins.

In any aspect or embodiment described herein, the substrate is selected from iron oxide nanoparticles, and crosslinked iron oxide nanoparticles, wherein the substrate is separated magnetically.

In any aspect or embodiment described herein, the substrate is selected from glass beads, crosslinked glass beads, and iron oxide nanoparticles crosslinked with glass beads, wherein the substrate is separated by gravity.

In certain aspects, the disclosure provides methods of recovering toxic cell particles from the blood of a cancer patient comprising the steps of: providing a blood sample from a cancer patient; incubating the blood sample with the non-hemolytic composition as described herein (e.g., for about 5 minutes); separating from the blood the non-hemolytic composition with cancer cells and/or toxic cell particles bound thereto; fixing the bound cells with absolute ethanol; and immuno-staining with cytokeratin (CK-18), leucocyte common antigen (CD-45) and nuclear-staining probe 4′,6-diamidino-2-phenylindole (DAPI) and imaging under fluorescence microscope.

In any aspect or embodiment described herein, the toxic cell particles are selected from CTCs, CTC clusters, cell-free nucleic acids (CfDNA), cancer cells associated nucleic acids (CtDNA), and exosomes.

In any aspect or embodiment described herein, the ligand is selected from anti-epithelial cell adhesion molecule antibody and transferrin.

In any aspect or embodiment described herein, the non-hemolytic composition is selected from iron oxide nanoparticles and crosslinked iron oxide nanoparticles and separated from blood by magnetic separation.

In any aspect or embodiment described herein, the non-hemolytic composition is selected from glass beads, crosslinked glass beads and glass beads crosslinked with iron oxide nanoparticles and separated from blood by gravity separation.

In certain aspects, the disclosure provides methods of destroying CTCs from blood comprising the steps of: providing a blood sample from a cancer patient; incubating the blood sample with the non-hemolytic composition of as described herein (e.g., for about 5 minutes); isolating from the blood the non-hemolytic composition with cancer cells and/or toxic cell particles bound thereto; incubating for about two hours with an anticancer drug; and confirming the destruction of the cancer cells by fixing the composition with absolute ethanol and immuno-stained with cytokeratin (CK-18), leucocyte common antigen (CD-45) and with nuclear-staining probe 4′,6-diamidino-2-phenylindole (DAPI) and imaging under fluorescence microscope.

In any aspect or embodiment described herein, the cancer cells are a circulating tumor cell (CTC). In any aspect or embodiment described herein, the anticancer drug is selected from Doxorubicin, Methotrexate, Paclitaxel, 5 Fluorouracil, Camptothecin and Cisplatin. In any aspect or embodiment described herein, the ligand is selected from anti-Epithelial Cell Adhesion Molecule—(EpCAM) antibody, and transferrin. In any aspect or embodiment described herein, the substrate is selected from iron oxide nanoparticles and crosslinked iron oxide nanoparticles and separated from blood by magnetic separation. In any aspect or embodiment described herein, the substrate is selected from glass beads, crosslinked glass beads, and glass beads crosslinked with iron oxide nanoparticles, wherein the substrate is separated from blood by gravity separation.

1. A composition comprising:
a substrate; and
a ligand, wherein the substrate is glass, iron oxide or a combination thereof, and the ligand is at least one of (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, poly(amidoamine) dendrimer, anti-Epithelial Cell Adhesion Molecule—(EpCAM) antibody, transferrin, bovine serum albumin, N-acetylglucosamine or a combination thereof, and wherein the composition is non-hemolytic.

2. The composition of claim 1, wherein the substrate is iron oxide nanoparticles in the size range of from about 10 nm to about 300 nm.

3. The composition of claim 1, wherein the substrate is glass.

4. The composition of claim 3, wherein the glass is in the form selected from glass beads, glass capillaries and glass cover slips.

5. The composition of claim 1, further including a functionalizing agent attached to the substrate covalently or non-covalently.

6. The composition of claim 5, wherein the ligand is the functionalizing agent, and is attached to the substrate non-covalently.

7. The composition of claim 5, wherein a first portion of the functionalizing agent is linked to a first ligand covalently, and a second portion of the functionalizing agent is linked to a second ligand covalently, wherein the first ligand and the second ligand are not the same.

8. The composition of claim 5, further including a spacer linked to the functionalizing agent.

9. The composition of claim 8, wherein the ligand is the spacer, and is attached to the functionalizing agent covalently.

10. The composition of claim 8, wherein the spacer is at least one of glutathione, citric acid, succinic acid, 12 amino dodecanoic acid, iminothiolane, poly (amidoamine) dendrimer, cysteine, glutaraldehyde, aspartic acid, mercaptoacetic acid, mercaptopropanoic acid, iminothiolane hydrochloride and dicarboxylic acids, polyimides, poly(amidoamine) (PAMAM), or a combination thereof.

11. The composition of claim 5, wherein the functionalizing agent is selected from (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), glutathione.

12. A composition comprising
a substrate;
a functionalizing agent;
a spacer; and
a ligand, wherein the substrate is glass, iron oxide or a combination thereof, and the ligand is at least one of (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, poly(amidoamine) dendrimer, anti-Epithelial Cell Adhesion Molecule—(EpCAM) antibody, transferrin, bovine serum albumin, N-acetylglucosamine or a combination thereof, and wherein the composition is non-hemolytic.

13. The composition of claim 12, wherein the functionalizing agent is at least one of (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), (3-mercaptopropyl)triethoxysilane (MPTES), glutathione or a combination thereof.

14. The composition of claim 12, wherein the spacer is at least one of glutathione, citric acid, succinic acid, 12 amino dodecanoic acid, iminothiolane, poly amidoamine dendrimer, cysteine, glutaraldehyde, aspartic acid, mercaptoacetic acid, mercaptopropanoic acid, iminothiolane hydrochloride, N-γ-maleimidobutyryl-oxysuccinimide ester (GMBS), N-γ-maleimidobutyryl-oxysulfosuccinimide ester (sulfo-GMBS), sulfo-N-hydroxysuccinimide ester (BS(PEG), (PEGylated bis(sulfosuccinimidyl)suberate) or a combination thereof.

15. The composition of claim 12, wherein the spacer is selected from dicarboxylic acids, aliphatic diamines, ω thio carboxylic acid, ω amino carboxylic acids, polyethylene glycol, poly(methacrylic acid) (PMA), poly oligo(ethylene glycol) methacrylate, diglycidyl ether, poly(N-isopropyl acrylaminde) (PNIPAM), poly w) (PLA), and Polyvinyl alcohol (PVA), in the number average molecular weight from 50 to 50000 kilo Dalton.

16. The composition of claim 12, wherein the ligand is selected from anti-epithelial cell adhesion Molecule antibody, transferrin, bovine serum albumin, and N-acetylglucosamine.

17. The composition of claim 12, wherein the substrate is crosslinked by a crosslinking agent.

18. The composition of claim 17, wherein the crosslinking agent is selected from (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, and iminothiolane.

19. The composition of claim 17, wherein the substrate is magnetic iron oxide particle crosslinked by a cross linking agent, wherein the crosslinked magnetic iron oxide particle does not cause interference during imaging.

20. The composition of claim 17, wherein the substrate is glass beads crosslinked by a cross linking agent.

21. The composition of claim 20, wherein the crosslinking agent is selected from (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, and iminothiolane.

22. The composition of claim 12, wherein the substrate comprises iron oxide and glass, which are crosslinked to each other by a crosslinking agent.

23. The composition of claim 22, wherein the crosslinking agent is selected from (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl) triethoxysilane (GPTES), (3-mercaptopropyl)triethoxysilane (MPTES), glutathione, and iminothiolane.

24. The composition of claim 23, wherein the iron oxide substrate and glass substrate, respectively, is linked to a different ligand.

25. A method of crosslinking iron oxide nanoparticles comprising the steps of:
functionalizing a first sample of iron oxide nanoparticles with a functionalizing agent (F1);
functionalizing a second sample of iron oxide nanoparticles with functionalizing agent (F2);
crosslinking the first and second samples of iron oxide nanoparticles with a crosslinking agent;
isolating the crosslinked iron oxide particles magnetically; and
purifying the product.

26. The method of claim 25 wherein F1 is selected from cysteine, glutathione, 12-aminododecanoic acid, (3-aminopropyl)triethoxysilane (APTES), (3-glycidyloxypropyl) trimethoxysilane (GPTMS), (3-mercaptopropyl)trimethoxysilane (MPTMS), and (3-glycidyloxypropyl)triethoxysilane (GPTES).

27. The method of claim 25, wherein F2 is selected from cysteine, glutathione, 12-aminododecanoic acid, (3-aminopropyl)triethoxysilane (APTES), (3-glycidyloxypropyl) trimethoxysilane (GPTMS), (3-mercaptopropyl)trimethoxysilane (MPTMS), and (3-glycidyloxypropyl)triethoxysilane (GPTES).

28. A method of crosslinking iron oxide nanoparticles comprising the steps of:
dispersing glutathione functionalized iron oxide nanoparticles in distilled water;
treating with N-(3-dimethyl aminopropyl)-N′-ethylcarbodiimide hydrochloride and N-hydroxysuccinimide; and
recovering glutathione cross-linked iron oxide particles magnetically.

29. A method of crosslinking glass beads comprising the steps of:
functionalizing a first sample of glass beads with a functionalizing agent (F1);
functionalizing a second sample of glass beads with a functionalizing agent (F2);
crosslinking the first and second samples of glass beads with a crosslinking agent;
isolating the crosslinked glass beads by gravity separation; and
purifying the product.

30. The method of claim 29 wherein F1 is selected from (3-aminopropyl)triethoxysilane (APTES), (3-glycidyloxypropyl)trimethoxysilane (GPTMS), (3-mercaptopropyl) trimethoxysilane (MPTMS), (3-glycidyloxypropyl)triethoxysilane (GPTES), (3-mercaptopropyl)triethoxysilane (MPTES), glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, and cysteine.

31. The method of claim 29, wherein F2 is selected from glutathione, mercaptopropanol, mercaptopropionic acid, 12-amino dodecanoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl) trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

32. A method of crosslinking glass beads comprising the steps of:
functionalizing glass beads with a functionalizing agent (F1);
reacting the glass beads with glutaraldehyde for about 4 hours at room temperature followed by the addition of a second sample of glass beads functionalized with a functionalizing agent (F2);
reacting for 4 hours at room temperature, washing with distilled water; and
recovering the crosslinked glass beads by drying.

33. The method of claim 32 wherein F1 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl) triethoxysilane (MPTES).

34. The method of claim 32, wherein F2 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

35. A method of crosslinking iron oxide nanoparticles with glass beads comprising the steps of:
treating iron oxide nanoparticles functionalized with a functionalizing agent (F1) with N-(3-dimethylaminopropyl)-N′-ethylcarbodiimide hydrochloride and N-hydroxysuccinimide in phosphate buffer of pH 6, and reacting the same for about 4 hours at room temperature;
adding the functionalized iron oxide nanoparticles to glass beads functionalized with a functionalizing agent (F2) in phosphate buffer of pH 7.2, and reacting the same for about 4 hours at room temperature and treating the reaction mixture with glutaraldehyde for about 4 hours;
purifying the product by washing with distilled water.

36. The method of claim 35, wherein F1 is selected from glutathione, mercaptopropanol, mercaptopropionic acid, 12-aminododeconoic acid, 3-amino-2-(hydroxymethyl)propanoic acid, 4-aminobutanoic acid, 3-amino-3-(4-nitrophenyl)propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl) triethoxysilane (MPTES).

37. The method of claim 35, wherein F2 is selected from glutathione, mercaptopropanol, mercaptopropionic acid, 12-aminododeconoic acid, 3-amino-2-(hydroxymethyl) propanoic acid, 4-aminobutanoic acid, 3-amino-3-(4-nitrophenyl) propionic acid, aminoethanoic acid, serine, cysteine, (3-aminopropyl)triethoxysilane (APTES), (3-glycidyloxypropyl)trimethoxysilane (GPTMS), (3-mercaptopropyl)trimethoxysilane (MPTMS), (3-glycidyloxypropyl)triethoxysilane (GPTES), and (3-mercaptopropyl)triethoxysilane (MPTES).

38. A method of recovering drugs from blood comprising the steps of:
admixing blood to the non-hemolytic composition of claim 1, wherein the ligand is bovine serum albumin;
incubating the admixture (e.g., for up to about 10 minutes);
separating the composition;
adding red blood cells lysis buffer;
washing the composition and adding sodium chloride buffer; and
analysing the supernatant for recovery of a drug by UV-Vis spectroscopy.

39. The method of claim 38, wherein the drug is selected from Vancomycin, Metformin, Doxorubicin, Methotrexate, Paclitaxel, 5 Fluorouracil, Cisplatin, Camptothecin, Docetaxel, Oxaliplatin, Cyclophosphamide and their pharmaceutically acceptable salts.

40. The method of claim 38, wherein the non-hemolytic composition substrate is selected from iron oxide nanoparticles, and crosslinked iron oxide nanoparticles, and wherein the non-hemolytic composition is separated by magnetic separation.

41. The method of claim 38, wherein the non-hemolytic composition substrate is selected from glass beads, cross linked glass beads, iron oxide nanoparticles crosslinked with glass beads, and wherein the non-hemolytic composition is separated by gravity separation.

42. A method of recovering deoxyribonucleic acid from blood comprising the steps of:
admixing blood to the non-hemolytic composition of claim 1, wherein the ligand comprises poly(amidoamine) dendrimer;
incubating the admixture (e.g., for up to about 10 minutes);
separating the substrate;
adding red blood cells lysis buffer;
washing the substrate; and
analysing the supernatant for deoxyribonucleic acid by UV-Vis spectroscopy.

43. The method of claim 42, wherein the substrate is selected from iron oxide nanoparticles, crosslinked iron oxide nanoparticles, and is separated by magnetic separation.

44. The method of claim 42, wherein the substrate is selected from glass beads, cross linked glass beads, iron oxide nanoparticles crosslinked with glass beads and is separated by gravity separation.

45. A method of recovering protein from blood comprising the steps of:
admixing blood to the non-hemolytic composition of claim 1, wherein the substrate comprises N-acetylglucosamine;
incubating the admixture (e.g., for up to about 10 minutes);
separating the substrate;
adding red blood cell lysis buffer;
adding sodium chloride buffer; and
measuring protein recovered from the supernatant by UV-Vis spectroscopy.

46. The method of claim 45, wherein the protein is selected from bovine serum albumin, Prealbumin (transthyretin), Alpha 1 antitrypsin, Alpha-1-acid glycoprotein, Alpha-1-fetoprotein, alpha 2-macroglobulin, Gamma globulins, Beta-2 microglobulin, Haptoglobin, Ceruloplasmin, Complement component 3, Complement component 4, C-reactive protein (CRP), Lipoproteins (chylomicrons, VLDL, LDL, HDL), Transferrin, Prothrombin, and Mannose-binding lectins.

47. The method of claim 45 wherein the substrate is selected from iron oxide nanoparticles, and crosslinked iron oxide nanoparticles, wherein the substrate is separated magnetically.

48. The method of claim 45 wherein the substrate is selected from glass beads, crosslinked glass beads, and iron oxide nanoparticles crosslinked with glass beads, wherein the substrate is separated by gravity.

49. A method of recovering toxic cell particles from the blood of a cancer patient comprising the steps of:
providing a blood sample from a cancer patient;
incubating the blood sample with the non-hemolytic composition of claim 1 (e.g., for about 5 minutes);
separating from the blood the non-hemolytic composition with cancer cells and/or toxic cell particles bound thereto;
fixing the bound cells with absolute ethanol; and
immuno-staining with cytokeratin (CK-18), leucocyte common antigen (CD-45) and nuclear-staining probe 4′,6-diamidino-2-phenylindole (DAPI) and imaging under fluorescence microscope.

50. The method of claim 49, wherein the toxic cell particles are selected from CTCs, CTC clusters, cell-free nucleic acids (CfDNA), cancer cells associated nucleic acids (CtDNA), and exosomes.

51. The method of claim 49, wherein the ligand is selected from anti-epithelial cell adhesion molecule antibody and transferrin.

52. The method of claim 49, wherein the non-hemolytic composition is selected from iron oxide nanoparticles and crosslinked iron oxide nanoparticles and separated from blood by magnetic separation.

53. The method of claim 49, wherein the non-hemolytic composition is selected from glass beads, crosslinked glass beads and glass beads crosslinked with iron oxide nanoparticles and separated from blood by gravity separation.

54. A method of destroying CTCs from blood comprising the steps of:
providing a blood sample from a cancer patient;
incubating the blood sample with the non-hemolytic composition of claim 1 (e.g., for about 5 minutes);
isolating from the blood the non-hemolytic composition with cancer cells and/or toxic cell particles bound thereto;
incubating for two hours with an anticancer drug; and
confirming the destruction of the cancer cells by fixing the composition with absolute ethanol and immuno-stained with cytokeratin (CK-18), leucocyte common antigen (CD-45) and with nuclear-staining probe 4′,6-diamidino-2-phenylindole (DAPI) and imaging under fluorescence microscope.

55. The method of claim 54, wherein the cancer cells are a circulating tumor cell (CTC).

56. The method of claim 54, wherein the anticancer drug is selected from Doxorubicin, Methotrexate, Paclitaxel, 5 Fluorouracil, Camptothecin and Cisplatin.

57. The method of claim 54, wherein the ligand is selected from anti-Epithelial Cell Adhesion Molecule—(EpCAM) antibody, and transferrin.

58. The method of claim 54, wherein the substrate is selected from iron oxide nanoparticles and crosslinked iron oxide nanoparticles and separated from blood by magnetic separation.

59. The method of claim 54, wherein the substrate is selected from glass beads, crosslinked glass beads, and glass beads crosslinked with iron oxide nanoparticles, wherein the substrate is separated from blood by gravity separation.
